(19) United States
(12) Patent Application Publication
(10) Pub. No.: US 2023/0313074 A1
(43) Pub. Date: Oct. 5, 2023

(54) WASHING AND CLEANING AGENTS HAVING IMPROVED ENZYME STABILITY

(71) Applicant: Henkel AG & Co. KGaA, Duesseldorf (DE)

(72) Inventors: Nina MUSSMANN, Willich (DE); Christian DEGERING, Erkrath (DE); Susanne WIELAND, Zons/Dormagen (DE); Thomas WEBER, Weimar (Lahn) (DE); Inken PRUESER, Duesseldorf (DE); Thorsten BASTIGKEIT, Wuppertal (DE)

(21) Appl. No.: 17/628,903

(22) PCT Filed: Jul. 16, 2020
(86) PCT No.: PCT/EP2020/070119

§ 371 Date: Jan. 21, 2022

(30) Foreign Application Priority Data
Jul. 22, 2019 (EP) 19187522.8

(51) Int. Cl. C11D 3/386; C11D 3/39; C11D 11/00

(52) U.S. Cl. CPC C11D 3/38618; C11D 11/0023; C11D 3/3937; C11D 3/38636

(57) ABSTRACT

Washing or cleaning agents may include at least one protease and at least one stabilizer compound. The protease(s) may have an amino acid sequence with at least 70% sequence identity with the amino acid sequence specified in SEQ ID NO:1 over the entire length thereof and has an amino acid substitution at at least one of the positions corresponding to the positions 12, 43, 122, 127, 154, 156, 160, 211, or 222, in each case based on the numbering according to SEQ ID NO:1. The stabilizer compound(s) may be selected from the group consisting of a phenylboronic acid derivative, boric acid, a peptide inhibitor, and combinations thereof.

### REFERENCE TO A SEQUENCE LISTING SUBMITTED VIA EFS-WEB

The content of the ASCII text file of the sequence listing named P83056US_SEQLTXT.txt”, which is 7 kb in size was created on Jul. 22, 2019; the sequence listing is incorporated by reference in its entirety. The sequence listing was corrected on Jul. 29, 2022 to correct the position location of “Xaa” where necessary and to correct the number of proteins in each sequence; the corrected sequence listing was electronically submitted via EFS-Web herewith; the corrected sequence listing is incorporated by reference in its entirety.

### CROSS-REFERENCE TO RELATED APPLICATIONS

The present application is a national stage entry according to 35 U.S.C. § 371 of PCT Application No. PCT/EP2020/070119 filed on Jul. 16, 2020; which claims priority to German Patent Application Serial No. 19187522.8 filed on Jul. 22, 2019; all of which are incorporated herein by reference in their entirety and for all purposes.

### TECHNICAL FIELD

The disclosure is in the field of enzyme technology. The disclosure relates to washing or cleaning agents comprising at least one Bacillus gibsonii protease and at least one stabilizer compound, wherein the stabilizer compound is selected from the group consisting of phenylboronic acid derivative, boric acid, peptide inhibitor, and combinations thereof. The disclosure also pertains to the corresponding washing and cleaning methods, the use of the agents described herein and the use of a stabilizer compound selected from the group consisting of phenylboronic acid derivative, boric acid, peptide inhibitor, and combinations thereof for improving the stability of a Bacillus gibsonii protease in washing or cleaning agents and/or further enzymes optionally contained in the washing or cleaning agent.

### BACKGROUND

The use of enzymes in washing and cleaning agents has been established in the prior art for decades. They are used to expand the performance range of the agents in question according to their special activities. These include in particular hydrolytic enzymes such as proteases, amylases, lipases and cellulases. The first three mentioned hydrolyze proteins, starch and fats and thus contribute directly to the removal of dirt. Cellulases are used in particular due to their effect on fabric. Another group of washing and cleaning agent enzymes are oxidative enzymes, in particular oxidases, which, optionally in conjunction with other components, are preferably used to bleach stains or to produce the bleaching agents in situ. In addition to these enzymes, which are subject to continuous optimization, other enzymes such as pectinases, ß-glucanases, mannanases or other hemicellulases (glycosidases) are constantly being made available for use in washing and cleaning agents in particular in order to be able to optimally tackle specific stains, to hydrolyze specific vegetable polymers in particular.

Proteases are the longest-established enzymes and are contained in virtually all modern, effective washing and cleaning agents. This makes them one of the technically most important enzymes of all. Of these, in turn, proteases of the subtilisin type (subtilases, subtilopeptidases, EC 3.4.21.62), which are serine proteases due to the catalytically active amino acids, are particularly important. They act as non-specific endopeptidases and hydrolyze any acid amide bonds that are inside peptides or proteins. Their optimum pH is usually in the distinctly alkaline range. An overview of this family is given, e.g., in the article “Subtilases: Subtilisin-like Proteases” by R. Siezen, pages 75-95 in “Subtilisin enzymes,” published by R. Bott and C. Betzel, New York, 1996. Subtilases are formed naturally from microorganisms. In particular, the subtilisins formed and secreted by Bacillus species are the most significant group of subtilases.

In washing and cleaning agents, proteases are used to break down protein-containing stains on the items to be cleaned. However, they also hydrolyze themselves (autoproteolysis) and all other proteins contained in the agents in question, i.e., in particular other enzymes contained in the washing and cleaning agents. This occurs particularly during the cleaning process, i.e., in the aqueous washing or cleaning liquor when comparatively favorable reaction conditions are present. To a lesser extent, however, this also occurs during storage of the agent in question, which is why long storage periods are always accompanied by a certain loss of protease activity and the activities of the other enzymes. As a result of the loss of enzymatic activity, the enzymes no longer demonstrate optimal cleaning performance. This is particularly problematic in gel or liquid and in particular in water-containing formulations because in this form both the reaction medium and the hydrolysis reagent are provided with the water contained.

In general, only selected proteases are suitable for use in liquid, surfactant-containing preparations in any case. Many proteases do not exhibit sufficient catalytic performance in such preparations or they are not sufficiently stable. For the use of proteases in cleaning agents, therefore, a high catalytic activity and stability under conditions as they are during a wash cycle is particularly desirable.

One goal in the development of washing and cleaning agent formulations is therefore that of stabilizing the enzymes contained, in particular during storage, and also to prevent them from denaturing and/or cleaving or breaking down and/or decomposing due to physical influences or oxidation, etc., in particular during the storage and/or use of the washing or cleaning agent. One focus of these developments is that of protecting the proteins and/or enzymes contained from (auto)proteolytic cleavage. This can be done by building up physical barriers, for example by encapsulating the enzymes in specific enzyme granules or by packaging the agents in two-chamber or multi-chamber systems. The other way, which is frequently used, consists in adding chemical compounds which inhibit the proteases and thus act in general as stabilizers for proteases and the other proteins and enzymes contained. However, these must be reversible protease inhibitors, since the protease activity should only be prevented temporarily, in particular during storage, but not during the cleaning process.

The prior art describes various reversible protease inhibitors, for example polyols, in particular glycerol and 1,2-propylene glycol, benzamidine hydrochloride, borax, boric acids, boronic acids or the salts or esters thereof. Using boric acid derivatives together with polyols is also known. 4-formylphenylboronic acid (4-FPBA) is also a protease inhibitor known from the prior art. Peptide aldehydes, i.e., oligopeptides having a reduced C-terminus, in particular those consisting of 2 to 50 monomers, are also described for this purpose. The reversible peptide protease inhibitors include, inter alia, ovomucoid and leupeptin. Specific, reversible peptide inhibitors and fusion proteins from proteases and specific peptide inhibitors are also used for this purpose.

However, there is still a need to improve the cleaning performance of enzyme-containing washing and cleaning agents and to better stabilize the enzymes contained in the washing and cleaning agents.

### SUMMARY

Surprisingly, it has now been found that a protease from Bacillus gibsonii that has at least 70% sequence identity with the amino acid sequence specified in SEQ ID NO:1 over the entire length thereof and has an amino acid substitution at at least one of the positions which correspond to the positions 12, 43, 122, 127, 154, 156, 160, 211, 212 and 222, in each case based on the numbering according to SEQ ID NO:1, can be better stabilized by a stabilizer compound selected from the group consisting of phenylboronic acid derivative, boric acid, peptide inhibitor, and combinations thereof than conventional proteases and is therefore particularly suitable for use in washing or cleaning agents.

Therefore, the object is, in a first aspect, a washing or cleaning agent comprising at least one Bacillus gibsonii protease which has at least 70% sequence identity with the amino acid sequence specified in SEQ ID NO:1 over the entire length thereof and has an amino acid substitution at at least one of the positions which correspond to the positions 12, 43, 122, 127, 154, 156, 160, 211, 212 and 222, in each case based on the numbering according to SEQ ID NO:1, and at least one stabilizer compound, wherein the stabilizer compound is selected from the group consisting of phenylboronic acid derivative, boric acid, a peptide inhibitor, and combinations thereof.

“Peptide inhibitor” is understood to mean a compound of general formula (I) or a compound of general formula (II), wherein the compound of formula (I) or (II) is optionally present together with a salt of formula (III).

The compound of formula (I) has the following structural formula:

Z-A-NH—CH(R)—C(O)—X (I),

where A is an amino acid functional group; X is hydrogen; Z is an N-capping functional group selected from phosphoramidate [(R′O)₂(O)P—], sulfenamide [(SR′)₂-], sulfonamide [(R′(O)₂S—], sulfonic acid [SO₃H], phosphinamide [(R)₂(O)P—], sulfamoyl derivatives [R′O(O)₂S—], thiourea [(R)₂N(O)C—], thiocarbamate [R′O(S)C—], phosphonate [R′—P(O)OH], amidophosphate [R′O(OH)(O)P—], carbamate (R′O(O)C—) and urea (R′NH(O)C—), where each R′ is independently selected from straight-chain or branched C₁-C₆ unsubstituted alkyl, phenyl, C₇-C₉ alkylaryl and cycloalkyl functional groups, where the cycloalkyl ring can be a C₄-C₈ cycloalkyl ring and can contain one or more heteroatoms selected from O, N, and S; and R is selected from straight-chain or branched C₁-C₆ unsubstituted alkyl, phenyl and C₇-C₉ alkylaryl functional groups; and stereoisomers, tautomers and salts thereof.

The compound of formula (II) has the following structural formula:

Y—B₁—B₀—X (II),

where X is hydrogen; B₁ is a single D or L amino acid functional group; B₀ is an amino acid functional group and Y consists of one or more, preferably one or two, amino acid functional groups and optionally consists of an N-capping functional group, wherein the N-capping functional group is as defined under (I).

The salt of formula (III) has the following structural formula:

(C^{E+})ₚ(D^{F-})_{q} (III),

where C is a cation selected from the group consisting of Al³⁺, Ca²⁺, Li⁺, Mg²⁺, Mn²⁺, Ni²⁺, K⁺, NR″₄⁺ and Na⁺, where each R″ represents, independently of one another, H or a linear or branched, substituted or unsubstituted alkyl group, aryl group or alkenyl group which can all optionally contain one or more heteroatom(s); E is an integer from 1 to 3 and corresponds to the valency of the cation; p corresponds to the number of cations in the salt; D is an anion selected from the group consisting of CH₃COO⁻, Br⁻, CO₃²⁻, CI⁻, C₃H₅O(COO)₃³⁻, HCOO⁻, HCO₃⁻, HSO₄⁻, C₂O₄²⁻, SO₄²⁻, and SO₃²⁻; F is an integer from 1 to 3 and corresponds to the valency of the anion; q corresponds to the number of anions in the salt; wherein the net charge on the salt is 0, i.e., ((E)·p)−((F)·q)=0 applies.

Preferred functional groups R are selected from methyl, iso-propyl, sec-butyl, iso-butyl, —C₆H₅, —CH₂—C₆H₅, and —CH₂—CH₂—C₆H₅ such that the part —NH—CH(R)—C(O)—X of the compound of formula (I) is derived from the amino acids Ala, Val, Ile, Leu, PGly (phenylglycine), Phe and HPhe (homophenylalanine) by the carboxyl group being converted into an aldehyde group or trifluoromethyl ketone group. Although such functional groups are therefore not amino acids (although they can be synthesized from an amino acid precursor), in the case of the enzyme stabilizers listed here by way of example, for the sake of simplicity, the aldehyde part of the inhibitors, which is derived from the corresponding amino acids, is denoted by the addition “H” after the analogous amino acid (e.g., “-AlaH” represents the functional group “—NHCH(CH₃)C(O)H”). Trifluoromethyl ketones are identified in the same way by the addition “CF₃” after the analogous amino acid (e.g., “-AlaCF₃” represents the functional group “—NHCH(CH₃)C(O)CF₃”).

“Phenylboronic acid derivative” is understood to mean a compound of formula (IV). The compound of formula (IV) has the following structural formula:

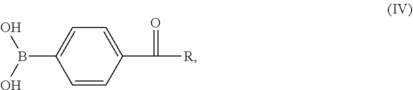

where R is hydrogen, a hydroxyl group, a C₁-C₆ alkyl group, a substituted C₁-C₆ alkyl group, a C₁-C₆ alkenyl group or a substituted C₁-C₆ alkenyl group.

A further objective is a washing or cleaning agent comprising at least one Bacillus gibsonii protease which has at least 70% sequence identity with the amino acid sequence specified in SEQ ID NO:1 over the entire length thereof and has at least one of the amino acid substitutions Q12L, I43V, M122L, D127P, N154S, T156A, G160S, M211N, M211L, P212D, P212H or A222S at at least one of the positions which correspond to the positions 12, 43, 122, 127, 154, 156, 160, 211, 212 and 222, in each case based on the numbering according to SEQ ID NO:1, and at least one stabilizer compound, wherein the stabilizer compound is selected from the group consisting of a phenylboronic acid derivative, boric acid, a peptide inhibitor, and combinations thereof.

Another objective is a method for producing such a washing or cleaning agent.

A further objective is the use of such a washing or cleaning agent for cleaning textiles and/or hard surfaces, in particular dishes.

Another objective is the use of a stabilizer compound selected from the group consisting of phenylboronic acid derivative, boric acid, peptide inhibitor, and combinations thereof for improving the stability of a Bacillus gibsonii protease in washing or cleaning agents and/or further enzymes optionally present in the washing or cleaning agent.

A further objective is the use of a stabilizer compound selected from the group consisting of phenylboronic acid derivative, boric acid, peptide inhibitor, and combinations thereof for improving the stability of a Bacillus gibsonii protease in washing or cleaning agents, wherein the protease has at least 70% sequence identity with the amino acid sequence specified in SEQ ID NO:1 over the entire length thereof and has an amino acid substitution at at least one of the positions which correspond to the positions 12, 43, 122, 127, 154, 156, 160, 211, 212 and 222, in each case based on the numbering according to SEQ ID NO:1, in particular a Bacillus gibsonii protease which has at least 70% sequence identity with the amino acid sequence specified in SEQ ID NO: 1 over the entire length thereof and has at least one of the amino acid substitutions Q12L, I43V, M122L, D127P, N154S, T156A, G160S, M211N, M211L, P212D, P212H or A222S at at least one of the positions which correspond to the positions 12, 43, 122, 127, 154, 156, 160, 211, 212 and 222, in each case based on the numbering according to SEQ ID NO:1, and/or a further enzyme optionally contained in the washing or cleaning agent.

These and other aspects, features, and advantages will become apparent to a person skilled in the art through studying the following detailed description and claims. Any feature from one aspect can be used in any other aspect. Furthermore, it will readily be understood that the examples contained herein are intended to describe and illustrate but not to limit the invention and that, in particular, the invention is not limited to these examples.

### DETAILED DESCRIPTION

Unless indicated otherwise, all percentages are indicated in terms of wt. %. Numerical ranges that are indicated in the format “from x to y” also include the stated values. If several preferred numerical ranges are indicated in this format, it is readily understood that all ranges that result from the combination of the various endpoints are also included. “At least one,” as used herein, means one or more, i.e., 1, 2, 3, 4, 5, 6, 7, 8, 9, 10, 11, 12, 13, 14 or more. The term “washing and cleaning agents” or “washing or cleaning agent,” as used herein, is synonymous with the term “agent” and denotes a composition for cleaning textiles and/or hard surfaces, in particular dishes, as explained in the description. “About,” “approx.” or “approximately,” as used herein in relation to a numerical value, relates to the corresponding numerical value±10%, preferably ±5%.

“Improving the stability of an enzyme” is when the presence of a stabilizer compound causes a washing or cleaning agent comprising at least one protease and at least one stabilizer compound (washing or cleaning agent) to have a higher enzymatic activity of the protease and/or optionally further enzymes contained in the washing or cleaning agent after storage compared to a control preparation which differs from the washing or cleaning agent only due to the absence of the stabilizer compound (control). After storage, the washing or cleaning agent therefore has a higher residual activity of the contained protease and/or optionally further contained enzymes compared to the control, with the washing or cleaning agent and the control having the same initial enzymatic activity at the start of storage and both agents being treated in the same way, in particular with regard to the storage conditions and the determination of the enzyme activity. Increasingly preferably, storage takes place for at least 1 week, 2 weeks, 3 weeks, 4 weeks and particularly preferably for 7 weeks. More preferably, storage takes place at a temperature of, increasingly preferably, 20° C., 25° C., 30° C. or 40° C.

The stabilizer compound used in washing or cleaning agents can be a peptide inhibitor of formula (I) or (II), as defined above.

The aldehydes of the peptide inhibitors, as used herein, can be produced from the corresponding amino acids, the C-terminal carboxyl group of the amino acid being converted to an aldehyde group. Such aldehydes can be prepared by means of known methods, as described, for example, in U.S. Pat. No. 5,015,627, EP0185930, EP0583534 and DE3200812.

The trifluoromethyl ketones, as used herein, can also be produced from the corresponding amino acids by the C-terminal carboxyl group being converted to a trifluoromethyl ketone group. Such trifluoromethyl ketones can be produced by means of known methods, as described, for example, in EP0583535.

In preferred embodiments, the substituent A is selected from Ala, Gly, Val, Ile, Leu, Phe and Lys.

The N-terminal end of the peptide inhibitor according to formula (I) and/or the peptide inhibitor according to formula (II) is protected by a protective group which caps the N-terminus, the group being selected from the group consisting of carbamates, ureas, sulfonamides, phosphonamides, thioureas, sulfenamides, sulfonic acids, phosphinamides, thiocarbamates, amidophosphates and phosphonamides. In a preferred embodiment, however, the N-terminal end is protected by a methyl group, ethyl group, or benzyl carbamate group [CH₃O—(O)C—; CH₃CH₂O—(O)C—; or C₆H₅CH₂O—(O)C—], a methyl group, ethyl group or benzylurea group [CH₃NH—(O)C—; CH₃CH₂NH—(O)C—; or C₆H₅CH₂NH—(O)C—], a methyl group, ethyl group or benzylsulfonamide group [CH₃SO₂—; CH₃CH₂SO₂—; or C₆H₅CH₂SO₂—], or a methyl group, ethyl group or benzyl amidophosphate group [CH₃O(OH)(O)P—; CH₃CH₂O(OH)(O)P—; or C₆H₅CH₂O(OH)(O)P—].

The synthesis of the N-capping groups can be carried out using methods known to a person skilled in the art, cf., for example, EP3263289 or the quotations cited therein.

In addition to a peptide inhibitor of formula (I) or (II), the agents can comprise salts of formula (III). These salts can be contained in a concentration of from 50 to 2000 mM, preferably from 70 to 1500 mM, more preferably from 100 to 1000 mM, even more preferably from 150 to 500 mM and more preferably of 200 mM. In further preferred embodiments, the salt of formula (III) is Na₂SO₄.

The term “alkyl,” as used herein, refers to an aliphatic hydrocarbon group which can be straight or branched and comprises 1 to 20 carbon atoms in the chain. The term “aryl,” as used herein, refers to an aromatic monocyclic or multicyclic ring system comprising 6 to 14 carbon atoms. The term “alkenyl,” as used herein, refers to an aliphatic hydrocarbon group which contains at least one carbon-carbon double bond and can be straight or branched and comprises 2 to 15 carbon atoms in the chain.

In preferred embodiments, B₀ is a D or L amino acid functional group selected from Tyr, m-tyrosine, 3,4-dihydroxyphenylalanine, Phe, Val, Met, Nva, Leu, Ile and Nle and/or B₁ is a D or L amino acid functional group having an (optionally substituted) small aliphatic pendent group, preferably Ala, Cys, Gly, Pro, Ser, Thr, Val, Nva or Nle. In further preferred embodiments, Y is B₂, B₃-B₂, Z—B₂, Z—B₃-B₂, where B₂ and B₃ are, in each case independently of one another, an amino acid functional group and Z is an N-capping functional group, wherein the N-capping functional group is as defined above. In further preferred embodiments, B₂ is selected from Val, Gly, Ala, Arg, Leu, Phe and Thr, and/or B₃ is selected from Phe, Tyr, Trp, phenylglycine, Leu, Val, Nva, Nle and Ile.

Unless otherwise stated, the amino acids in the above formulas are linked via peptide bonds and all peptides or peptide-like compounds are always shown from the N-terminus to the C-terminus, unless otherwise stated.

The agents can contain a peptide inhibitor of formula (I) and, alternatively or in addition to a peptide inhibitor of formula (I), a peptide inhibitor of formula (II).

The agents can contain the peptide inhibitor of formula (I) and/or (II) in a concentration of from 0.01 to 50 mM, preferably from 0.05 to 5 mM and more preferably from 0.1 to 0.5 mM. If a plurality of peptide inhibitors of formulas (I) and/or (II) are contained, these details refer to the total concentration.

Examples of peptide inhibitors of formulas (I) and (II) which can be used comprise, but are not limited to Cbz-Arg-Ala-Tyr-H, Ac-Gly-Ala-Tyr-H, Cbz-Gly-Ala-Tyr-H, Cbz-Gly-Ala-Tyr-H, Cbz-Val-Ala-Tyr-H, Cbz-Gly-Ala-Phe-H, Cbz-Gly-Ala-Val-H, Cbz-Gly-Gly-Tyr-H, Cbz-Gly-Gly-Phe-H, Cbz-Arg-Val-Tyr-H, Cbz-Leu-Val-Tyr-H, Ac-Leu-Gly-Ala-Tyr-H, Ac-Phe-Gly-Ala-Tyr-H, Ac-Tyr-Gly-Ala-Tyr-H, Ac-Phe-Gly-Ala-Leu-H, Ac-Phe-Gly-Ala-Phe-H, Ac-Phe-Gly-Val-Tyr-H, Ac-Phe-Gly-Ala-Met-H, Ac-Trp-Leu-Val-Tyr-H, MeO-CO-Val-Ala-Leu-H, MeNCO-Val-Ala-Leu-H, MeO-CO-Phe-Gly-Ala-Leu-H, MeO-CO-Phe-Gly-Ala-Phe-H, MeSO₂-Phe-Gly-Ala-Leu-H, MeSO₂-Val-Ala-Leu-H, PhCH₂O(OH)(O)P-Val-Ala-Leu-H, EtSO₂-Phe-Gly-Ala-Leu-H, PhCH₂SO₂-Val-Ala-Leu-H, PhCH₂O(OH)(O)P-Leu-Ala-Leu-H, PhCH₂O(OH)(O)P-Phe-Ala-Leu-H, MeO(OH)(O)P-Leu-Gly-Ala-Leu-H, α-MAPI, ß-MAPI, Phe-urea-Arg-Val-Tyr-H, Phe-urea-Gly-Gly-Tyr-H, Phe-urea-Gly-Ala-Phe-H, Phe-urea-Gly-Ala-Tyr-H, Phe-urea-Gly-Ala-Leu-H, Phe-urea-Gly-Ala-Nva-H, Phe-urea-Gly-Ala-Nle-H, Tyr-urea-Arg-Val-Tyr-H, Tyr-urea-Gly-Ala-Tyr-H, Phe-Cys-Ser-Arg-Val-Phe-H, Phe-Cys-Ser-Arg-Val-Tyr-H, Phe-Cys-Ser-Gly-Ala-Tyr-H, Antipain, GE20372A, GE20372B, chymostatin A, chymostatin B and chymostatin C.

As used herein, the term “Cbz” refers to the benzyloxycarbonyl group having the empirical formula C₇H₇O. This is used as a protective group. Further terminal groups in the peptide inhibitors can be: “Ph:” phenyl; “Ac:” acetyl; and “Me:” methyl. The term “urea,” as used herein, is synonymous with carbamide.

In various embodiments, all stereoisomers, in particular enantiomers and diastereomers, tautomers and salts of the compounds described above may be used herein.

Without wishing to be bound by theory, it is assumed that the addition of a salt of formula (III) to a peptide inhibitor of formula (I) or (II) further stabilizes the enzyme-peptide inhibitor complex by removing free reactive water molecules. This increases the binding efficiency of the peptide inhibitor to the enzyme and/or increases the ionic strength, as a result of which the enzyme-peptide inhibitor complex is ultimately stabilized. By using at least one salt of formula (III), it is possible to use the peptide inhibitors in moderate concentrations (0.01 to 50 mM). The protease and optionally further contained proteins, in particular other enzymes, are in this way protected from proteolysis (stabilized against proteolysis) by this enzyme, in particular proteases, and are thus fully effective even after storage.

Furthermore, the compounds have good water solubility, and therefore they can be easily incorporated into corresponding agents and precipitation during storage is avoided.

The agents can contain a peptide inhibitor of formula (I) and/or a peptide inhibitor of formula (II). Alternatively and/or in addition to a peptide inhibitor of formula (I) and/or (II), the agents can contain a phenylboronic acid derivative and/or boric acid.

The stabilizer compound used in washing or cleaning agents can be boric acid. In a washing or cleaning agent, the boric acid is preferably contained in an amount of from 0.05 to 5.5 wt. % and increasingly preferably from 0.075 to 4.5 wt. %, from 0.09 to 3.5 and from 0.1 to 2.49 wt. %.

The stabilizer compound used in washing or cleaning agents can be a phenylboronic acid derivative of formula (IV):

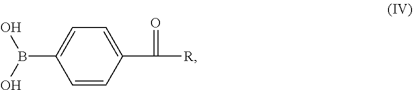

where R is hydrogen, a hydroxyl group, a C₁-C₆ alkyl group, a substituted C₁-C₆ alkyl group, a C₁-C₆ alkenyl group or a substituted C₁-C₆ alkenyl group.

In a preferred embodiment, the functional group R in the phenylboronic acid derivative is a C₁-C₆ alkyl group and, among these, is more preferably —CH₃, —CH₃CH₂ or —CH₃CH₂CH₂. In a further preferred embodiment, the functional group R in the phenylboronic acid derivative is hydrogen. In a very particularly preferred embodiment, the phenylboronic acid derivative is 4-formylphenylboronic acid (4-FPBA). The proportion by weight of 4-formylphenylboronic acid with respect to the total weight of the washing or cleaning agent is preferably from 0.0005 to 2.0 wt. %, preferably from 0.001 to 1.0 wt. %, more preferably from 0.01 to 0.5 wt. % and even more preferably from 0.02 to 0.2 wt. %.

Phenylboronic acid derivatives which can be used can also have further chemical modifications on the phenyl ring, in particular they can contain one or more methyl, amino, nitro, chloro, fluoro, bromo, hydroxyl, formyl, ethyl, acetyl, t-butyl, anisyl, benzyl, trifluroacetyl, N-hydroxysuccinimide, t-butyloxycarbonyl, benzoyl, 4-methylbenzyl, thioanizyl, thiocresyl, benzyloxymethyl, 4-nitrophenyl, benzyloxycarbonyl, 2-nitrobenzoyl, 2-nitrophenylsulphenyl, 4-toluenesulphonyl, pentafluorophenyl, diphenylmethyl, 2-chlorobenzyloxycarbonyl, 2,4,5-trichlorophenyl, 2-bromobenzyloxycarbonyl, 9-fluorenylmethyloxycarbonyl, triphenylmethyl, 2,2,5,7,8-pentamethyl-chroman-6-sulfonyl functional groups or groups, or combinations thereof.

All compounds which are provided as stabilizer compounds can be present in the washing or cleaning agent in all protonated or deprotonated forms. Furthermore, all such compounds, in particular their deprotonated forms, can be associated with cations. Preferred cations in this regard are monovalent or polyvalent, in particular divalent, cations, in particular Na ions (Na⁺), K ions (K⁺), Li ions (Li⁺), Ca ions (Ca²⁺), Mg ions (Mg²⁺), Mn ions (Mn²⁺) and Zn ions (Zn²⁺). Na ions (Nat) are particularly preferred.

In addition to the stabilizer compounds mentioned, an agent can contain at least one further stabilizer, in particular a polyol, such as glycerol or 1,2-ethylene glycol, and/or an antioxidant in further embodiments. In preferred embodiments, the interaction of the stabilizer compounds, in particular the interaction of boric acid, 4-FPBA and peptide inhibitor, results in a synergistic enzyme stabilization. This is understood to mean improved enzyme stabilization by means of the combination of the compounds in comparison with enzyme stabilization by means of each one of these compounds alone and also in comparison with the sum of the individual performances of the compounds with regard to enzyme stabilization.

In washing or cleaning agents, which in one embodiment are predominantly in solid form and in another embodiment are predominantly in liquid, pasty or gel form, the washing or cleaning agent comprises the enzyme, i.e., the protease, based in each case on the total weight of the washing or cleaning agent, in an amount of from 0.005 to 5 wt. %, preferably from 0.05 to 2 wt. %, more preferably from 0.01 to 0.5 wt. %, and even more preferably from 0.02 to 0.2 wt. %, and, if the at least one stabilizer compound is a peptide inhibitor, it comprises said peptide inhibitor in an amount of from 0.01 to 15 wt. %, preferably from 0.05 to 5 wt. %, more preferably from 0.1 to 1 wt. %, and even more preferably from 0.2 to 0.75 wt. %; and/or if the at least one stabilizer compound is a phenylboronic acid derivative, in particular 4-FPBA, it comprises said phenylboronic acid derivative in an amount of from 0.0005 to 2.0 wt. %, preferably from 0.001 to 1.0 wt. %, more preferably from 0.01 to 0.5 wt. %, and even more preferably from 0.02 to 0.2 wt. %; and/or if the at least one stabilizer compound is boric acid, it comprises said boric acid in an amount of from 0.05 to 5.5 wt. %, preferably from 0.075 to 4.5 wt. %, more preferably from 0.09 to 3.5 wt. %, and even more preferably from 0.1 to 2.49 wt. %.

In various embodiments, the enzyme and the stabilizer compound can be pre-formulated in an enzyme composition. As is clear from the preceding remarks, the enzyme protein forms only a fraction of the total weight of conventional enzyme preparations. Protease preparations that are preferably used contain between 0.1 and 40 wt. %, preferably between 0.2 and 30 wt. %, particularly preferably between 0.4 and 20 wt. %, and in particular between 0.8 and 10 wt. %, of the enzyme protein. In such compositions, the stabilizer compound can be contained in an amount of from 0.05 to 35 wt. %, preferably from 0.05 to 10 wt. %, based on the total weight in the enzyme composition. This enzyme composition can then be used in washing or cleaning agents, specifically in amounts which lead to the final concentrations in the washing or cleaning agent indicated above.

The inventors have surprisingly found that a Bacillus gibsonii protease which has at least 70% sequence identity with the amino acid sequence specified in SEQ ID NO:1 over the entire length thereof and has at least one amino acid substitution at at least one of the positions which correspond to the positions 12, 43, 122, 127, 154, 156, 160, 211, 212 or 222 of the protease from Bacillus gibsonii according to SEQ ID NO:1, in particular at least one amino acid substitution selected from the group consisting of Q12L, I43V, M122L, D127P, N154S, T156A, G160S, M211N, M211L, P212D, P212H or A222S, can be stabilized better in washing or cleaning agents by a stabilizer compound selected from the group consisting of phenylboronic acid derivative, boric acid, peptide inhibitor, and combinations thereof than by conventional proteases.

This is particularly surprising insofar as none of the stabilizer compounds mentioned has previously been associated with improved stability of such a Bacillus gibsonii protease in washing or cleaning agents. Furthermore, none of the stabilizer compounds mentioned has previously been associated with improved stability of further enzymes contained in the washing or cleaning agent if said stabilizer compounds are present in the washing or cleaning agent together with the Bacillus gibsonii protease mentioned.

The proteases exhibit enzymatic activity, i.e., they are capable of hydrolyzing peptides and proteins, in particular in washing or cleaning agents. A protease used is therefore an enzyme which catalyzes the hydrolysis of amide/peptide bonds in protein/peptide substrates and is thus able to cleave proteins or peptides. Furthermore, a protease used is preferably a mature protease, i.e., the catalytically active molecule without signal peptide(s) and/or propeptide(s). Unless stated otherwise, the sequences specified also each refer to mature (processed) enzymes.

In various embodiments, the protease is a free enzyme. This means that the protease can act directly with all the components of an agent and, if the agent is a liquid agent, that the protease is in direct contact with the solvent of the agent (e.g., water). In other embodiments, an agent may contain proteases that form an interaction complex with other molecules or that contain a “coating.” In this case, an individual protease molecule or a plurality of protease molecules may be separated from the other components of the agent by a surrounding structure. Such a separating structure may arise from, but is not limited to, vesicles such as a micelle or a liposome. The surrounding structure may also be a virus particle, a bacterial cell or a eukaryotic cell. In various embodiments, an agent may include cells of Bacillus gibsonii or Bacillus subtilis which express the proteases, or cell culture supernatants of such cells.

Furthermore, in various embodiments, the Bacillus gibsonii protease used contains at least one amino acid substitution selected from the group consisting of Q12L, I43V, M122L, D127P, N154S, T156A, G160S, M211N, M211L, P212D, P212H or A222S, in each case based on the numbering according to SEQ ID NO:1. In further preferred embodiments, the protease used contains one of the following amino acid substitution variants: (i) I43V; (ii) M122L, N154S and T156A; (iii) M211N and P212D; (iv) M211L and P212D; (v) G160S; (vi) D127P, M211L and P212D; (vii) P212H; or (viii) Q12L, M122L and A222S, wherein the numbering in each case is based on the numbering according to SEQ ID NO: 1.

In a further embodiment, the protease used comprises an amino acid sequence which is preferably at least 70% and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5% and 98.8% identical to the amino acid sequence specified in SEQ ID NO:1 over the entire length thereof, and has one or more amino acid substitutions 12L, 43V, 122L, 127P, 154S, 156A, 160S, 211N, 211L, 212D, 212H or 222S at at least one of the positions which correspond to the positions 12, 43, 122, 127, 154, 156, 160, 211, 212 or 222 in the numbering according to SEQ ID NO:1.

The feature whereby a protease has the specified substitutions means that it contains at least one of the corresponding amino acids at the corresponding positions, i.e., not all of the 10 positions are otherwise mutated or deleted, for example by fragmentation of the protease.

Advantageous positions for sequence modifications, in particular substitutions, of the protease from Bacillus gibsonii that are of particular significance when transferred to homologous positions of the proteases used and impart advantageous functional properties to the protease are therefore the positions which correspond in an alignment to the positions 12, 43, 122, 127, 154, 156, 160, 211, 212 and 222 in SEQ ID NO:1, i.e., in the numbering according to SEQ ID NO:1. At the positions mentioned, the following amino acid functional groups are present in the wild-type molecule of the protease from Bacillus gibsonii: Q12, I43, M122, D127, N154, T156, G160, M211, P212 and A222.

The identity of nucleic acid sequences or amino acid sequences is determined by a sequence comparison. This sequence comparison is based on the BLAST algorithm established and commonly used in the prior art (cf., e.g., Altschul et al. (1990) “Basic local alignment search tool,” J. Mol. Biol. 215:403-410, and Altschul et al. (1997): “Gapped BLAST and PSI-BLAST: a new generation of protein database search programs,” Nucleic Acids Res., 25:3389-3402) and occurs in principle by similar sequences of nucleotides or amino acids in the nucleic acid or amino acid sequences being associated with one another. A tabular association of the positions concerned is referred to as alignment. Another algorithm available in the prior art is the FASTA algorithm. Sequence comparisons (alignments), in particular multiple sequence comparisons, are created using computer programs. The Clustal series (cf., e.g., Chenna et al. (2003) “Multiple sequence alignment with the Clustal series of programs,” Nucleic Acid Res. 31: 3497-3500), T-Coffee (c.f., e.g., Notredame et al. (2000) “T-Coffee: A novel method for multiple sequence alignments,” J. Mol. Biol. 302:205-217) or programs based on these programs or algorithms, for example, are frequently used. Sequence comparisons (alignments) using the computer program Vector NTI® Suite 10.3 (Invitrogen Corporation, 1600 Faraday Avenue, Carlsbad, California, USA) with the predetermined standard parameters, the AlignX module of which program for the sequence comparisons is based on ClustalW, are also possible. Unless stated otherwise, the sequence identity specified herein is determined by the BLAST algorithm.

Such a comparison also allows conclusions to be drawn regarding the similarity of the compared sequences. It is usually indicated in percent identity, i.e., the proportion of identical nucleotides or amino acid functional groups at the same positions or in an alignment of corresponding positions. The broader concept of homology takes conserved amino acid exchanges into account in the case of amino acid sequences, i.e., amino acids having similar chemical activity, since they usually perform similar chemical activities within the protein. Therefore, the similarity of the compared sequences may also be stated as percent homology or percent similarity. Identity and/or homology information can be provided regarding whole polypeptides or genes or only regarding individual regions. Homologous or identical regions of different nucleic acid or amino acid sequences are therefore defined by matches in the sequences. Such regions often have identical functions. They can be small and comprise only a few nucleotides or amino acids. Often, such small regions perform essential functions for the overall activity of the protein. It may therefore be expedient to relate sequence matches only to individual, optionally small regions. Unless stated otherwise, however, identity or homology information in the present application relates to the entire length of the particular nucleic acid or amino acid sequence indicated. The indication that an amino acid position corresponds to a numerically designated position in SEQ ID NO:1 therefore means that the corresponding position is associated with the numerically designated position in SEQ ID NO:1 in an alignment as defined above.

A further objective is a washing or cleaning agent comprising at least one protease and at least one of the above-mentioned stabilizer compounds, characterized in that the protease can be obtained from a protease as the starting molecule by single or multiple conservative amino acid substitution. The term “conservative amino acid substitution” means the exchange (substitution) of one amino acid functional group for another amino acid functional group, with this exchange not resulting in a change to the polarity or charge at the position of the exchanged amino acid, e.g., the exchange of a nonpolar amino acid functional group for another nonpolar amino acid functional group. Conservative amino acid substitutions comprise, for example: G=A=S, 1=V=L=M, D=E, N=Q, K=R, Y=F, S=T, G=A=I=V=L=M=Y=F=W=P=S=T.

Another objective is a washing or cleaning agent comprising at least one protease and at least one of the above-mentioned stabilizer compounds, characterized in that the protease can be obtained from a protease as a starting molecule by fragmentation, deletion mutagenesis, insertion mutagenesis or substitution mutagenesis and comprises an amino acid sequence which matches the starting molecule over a length of at least 50, 60, 70, 80, 90, 100, 110, 120, 130, 140, 150, 160, 170, 180, 190, 200, 210, 220, 230, 240, 250, 260, 261, 262, 263, 264, 265, 266, 267, 268 or 269 contiguous amino acids.

For instance, it is possible to delete individual amino acids at the termini or in the loops of the enzyme without the proteolytic activity being lost or diminished in the process. Furthermore, such fragmentation or deletion, insertion or substitution mutagenesis can also for example reduce the allergenicity of the enzymes in question and thus improve their overall applicability. Advantageously, the enzymes retain their proteolytic activity even after mutagenesis, i.e., their proteolytic activity corresponds at least to that of the starting enzyme. Substitutions can also exhibit advantageous effects. Both single and multiple contiguous amino acids can be exchanged for other amino acids.

A protease can additionally be stabilized, in particular by one or more mutations, for example substitutions, or by coupling to a polymer. An increase in stability during storage and/or during use, for example in the cleaning process, leads to longer enzymatic activity and thus improves the cleaning performance. In principle, all stabilization options which are described in the prior art and/or are appropriate are considered. Those stabilizations are preferred which are achieved by mutations of the enzyme itself, since such stabilizations do not require any further work steps following the recovery of the enzyme. Further possibilities for stabilization are, e.g.:
altering the binding of metal ions, in particular the calcium binding sites, for example by exchanging one or more of the amino acid(s) that are involved in the calcium binding with one or more negatively charged amino acids and/or by introducing sequence alterations in at least one of the sequences of the two amino acids arginine and glycine;
protecting against the influence of denaturing agents such as surfactants by mutations that cause an alteration of the amino acid sequence on or at the surface of the protein;
exchanging amino acids near the N-terminus with those likely to contact the rest of the molecule via non-covalent interactions, thus contributing to the maintenance of the globular structure.

Preferred embodiments are those in which the enzyme is stabilized in several ways since a plurality of stabilizing mutations act additively or synergistically.

The protease may have at least one chemical modification. A protease having such an alteration is referred to as a derivative, i.e., the protease is derivatized. Derivatives, within the meaning of the present application, shall thus be understood to mean those proteins in which the pure amino acid chain has been chemically modified. Such derivatizations can be achieved, e.g., in vivo by the host cell that expresses the protein. In this regard, couplings of low-molecular-weight compounds such as lipids or oligosaccharides are particularly noteworthy. However, the derivatizations may also be carried out in vitro, for example by the chemical conversion of a side chain of an amino acid or by covalent bonding of another compound to the protein. For example, it is possible to couple amines to carboxyl groups of an enzyme in order to alter the isoelectric point. Another such compound may also be another protein that is bound to a protein via bifunctional chemical compounds, for example. Derivatization is likewise understood to mean the covalent bonding to a macromolecular carrier or also a non-covalent inclusion in suitable macromolecular cage structures. Derivatizations may, e.g., affect the substrate specificity or bonding strength to the substrate or cause a temporary blockage of the enzymatic activity when the coupled substance is an inhibitor. This can be expedient, e.g., for the period of storage. Such modifications may further affect the stability or enzymatic activity. They can also be used to reduce the allergenicity and/or immunogenicity of the protein and for example increase its skin compatibility. For example, couplings with macromolecular compounds, for example polyethylene glycol, can improve the protein in terms of stability and/or skin compatibility. Derivatives of a protein can also be understood in the broadest sense to mean preparations of these proteins. Depending on the recovery, processing or preparation, a protein can be socialized with various other substances, e.g., from the culture of the producing microorganisms. A protein may also have been deliberately added to other substances, e.g., to increase its storage stability. This is also irrespective of whether or not it actually exhibits this enzymatic activity in a particular preparation. This is because it may be desired that it has no or only low activity during storage and exhibits its enzymatic function only at the time of use. This can be controlled via appropriate accompanying substances, for example.

Numerous proteases and in particular subtilisins are formed as so-called preproteins, i.e., together with a propeptide and a signal peptide, where the function of the signal peptide is usually to ensure the release of the protease from the cell producing it into the periplasm or the medium surrounding the cell, and the propeptide is usually necessary for the protease to fold correctly. The signal peptide and the propeptide are usually the N-terminal part of the preprotein. The signal peptide is cleaved off from the rest of the protease under natural conditions by a signal peptidase. The correct final folding of the protease, supported by the propeptide, then takes place. The protease is then in its active form and cleaves off the propeptide itself. After the propeptide has been cleaved off, the then-mature protease, in particular subtilisin, carries out its catalytic activity without the N-terminal amino acids originally present. For technical applications in general, the mature proteases, i.e., the enzymes processed after their production, are preferred over the preproteins. The proteases can also be modified by the cells producing them after the production of the polypeptide chain, for example by attaching sugar molecules, formylations or aminations, etc. Such modifications are post-translational modifications and can, but do not have to, have an influence on the function of the protease.

“Variant,” as used herein, refers to naturally or artificially generated variations of a native protease which has an amino acid sequence which is modified from the reference form. In addition to the amino acid alterations discussed above, proteases can have other amino acid alterations, in particular amino acid substitutions, insertions or deletions. Such proteases are, for example, developed by targeted genetic alteration, i.e., by mutagenesis methods, and optimized for specific applications or with regard to specific properties (for example with regard to their catalytic activity or stability, etc.). Furthermore, nucleic acids can be introduced into recombination approaches and can thus be used to generate completely new types of proteases or other polypeptides. The aim is to introduce targeted mutations such as substitutions, insertions or deletions into the known molecules in order, for example, to improve the cleaning performance of enzymes. For this purpose, in particular the surface charges and/or the isoelectric point of the molecules and thus their interactions with the substrate can be altered. For instance, the net charge of the enzymes can be altered in order to influence the substrate binding, in particular for use in washing and cleaning agents. Alternatively or additionally, one or more corresponding mutations can increase the stability or catalytic activity of the enzyme and thus improve its cleaning performance. Advantageous properties of individual mutations, e.g., individual substitutions, can complement one another. A protease that has already been optimized with regard to certain properties can therefore be developed, for example with regard to its stability towards surfactants and/or bleaching agents and/or other components.

For the description of substitutions relating to exactly one amino acid position (amino acid exchanges), the following convention is used herein: first, the naturally occurring amino acid is designated in the form of the internationally used one-letter code, followed by the associated sequence position and finally the inserted amino acid. A plurality of exchanges within the same polypeptide chain are separated by slashes. For insertions, additional amino acids are named following the sequence position. In the case of deletions, the missing amino acid is replaced by a symbol, for example a star or a dash, or a Δ is indicated before the corresponding position. For example, P14H describes the substitution of proline at position 14 by histidine, P14HT the insertion of threonine after the amino acid histidine at position 14 and P14* or ΔP14 the deletion of proline at position 14. This nomenclature is known to a person skilled in the art of enzyme technology.

The amino acid positions are in this case defined by an alignment of the amino acid sequence of a protease used with the amino acid sequence of the protease from Bacillus gibsonii, as indicated in SEQ ID NO:1. Furthermore, the assignment of the positions depends on the mature protein. This assignment is also to be used in particular if the amino acid sequence of a protease used comprises a higher number of amino acid functional groups than the protease from Bacillus gibsonii according to SEQ ID NO:1. Proceeding from the above-mentioned positions in the amino acid sequence of the protease from Bacillus gibsonii, the alteration positions in a protease used are those which are assigned to precisely these positions in an alignment.

In a further embodiment, the protease used is characterized in that the cleaning performance thereof is not significantly reduced compared with that of a protease comprising an amino acid sequence which corresponds to the amino acid sequence given in SEQ ID NO:1, i.e., has at least 80% of the reference washing performance, preferably at least 100%, more preferably at least 110% or more.

Washing or cleaning performance is understood to mean the ability of a washing or cleaning agent to partly or completely remove existing dirt. Both the washing or cleaning agent, which comprises the protease, or the washing or cleaning liquor formed by this agent, and the protease itself, have a cleaning performance. The cleaning performance of the protease thus contributes to the cleaning performance of the agent or the washing or cleaning liquor formed by the agent.

Washing or cleaning liquor is understood to mean the solution containing the washing or cleaning agent which acts on the textiles or hard surfaces and thus comes into contact with the stains present on the textiles or hard surfaces. The washing or cleaning liquor is usually created when the washing or cleaning process begins and the washing or cleaning agent is diluted with water, for example in a washing machine or dishwasher or in another suitable container. The cleaning performance can be determined in a system which contains an automatic dishwashing agent in a dosage as specified herein as well as the protease, wherein the proteases to be compared are used in the same concentration (based on active protein) and the cleaning performance with regard to tea, meat, spaghetti and/or creme brûlée stains is determined according to the IKW method in a Miele GSL (program 45° C., 21° dH). The concentration of the protease in the agent intended for this washing system is from 0.001 to 0.1 wt. %, preferably 0.01 to 0.06 wt. %, based on active, purified protein. A liquid reference agent (two-component formulation) for such a washing system can be composed as follows:

| | Active substance |
|---|---|
| Enzyme phase (EP)-Preparation A | content in wt. % |
| Phosphonate (e.g., HEDP), if permitted | 0.00-7.50 |
| by regulations | |
| CaCl₂ | 0.05-1.50 |
| Amylase-containing enzyme | 0.00-4.00 |
| composition (tq) | |
| Protease-containing enzyme | 0.00001-10 |
| composition (tq) | |
| Sorbitol | 2.00-10.00 |
| Sulfonic acid group-containing polymer | 0.00-12.00 |
| Thickener (based on acrylate or | 0.01-6.00 |
| xanthan gum) | |
| GLDA or MGDA | 3.00-25.00 |
| KOH | 0.50-4.00 |
| Non-ionic surfactants | 1.00-6.00 |
| Sodium citrate | 2.00-20.00 |
| Zinc salt | 0.00-1.00 |
| Remainder (perfume, dyes, preservatives, | to make up to 100 |
| water, enzyme stabilizer) (wt. %) | |

| | Active substance |
|---|---|
| Alkali phase (AP)-Preparation B | content in wt. % |
| Phosphonates, if permitted by | 0.00-7.50 |
| regulations | |
| Thickener (acrylate or xanthan gum) | 0.01-6.00 |
| GLDA or MGDA | 3.00-25.00 |
| KOH | 0.50-4.00 |
| Soda | 5.00-20.00 |
| Monoethanolamine | 0.00-5.00 |
| Acrylate polymer | 0.00-3.00 |
| Sodium citrate | 2.00-20.00 |
| Remainder (perfume, dyes, | to make up |
| preservatives, water, etc.) (wt. %) | to 100 |

The activity-equivalent use of the relevant protease ensures that the respective enzymatic properties, for example the cleaning performance on certain stains, are compared even if the ratio of active substance to total protein (the values of the specific activity) diverges. In general, a low specific activity can be compensated for by adding a larger amount of protein. Furthermore, the enzymes to be examined can also be used in the same amount of substance or amount by weight if the enzymes to be examined have a different affinity for the test substrate in an activity test. The expression “same amount of substance” in this context relates to a molar use of the enzymes to be examined. The expression “equal weight” relates to the use of the same weight of the enzymes to be examined.

Otherwise, methods for determining protease activity are well known to, and routinely used by, a person skilled in the art of enzyme technology. For example, such methods are disclosed in Tenside, vol. 7 (1970), pp. 125-132. Alternatively, the protease activity can be determined by the release of the chromophore para-nitroaniline (pNA) from the substrate suc-L-Ala-L-Ala-L-Pro-L-Phe-p-nitroanilide (AAPF). The protease cleaves the substrate and releases pNA. The release of the pNA causes an increase in absorbance at 410 nm, the temporal progression of which is a measure of the enzymatic activity (cf. Del Mar et al., 1979). The measurement is carried out at a temperature of 25° C., a pH of 8.6, and a wavelength of 410 nm. The measuring time is 5 min and the measuring interval is 20 to 60 s. The protease activity is usually indicated in protease units (PE). Suitable protease activities are, for example, 2.25, 5 or 10 PU per mL of washing liquor or per washing process. However, the protease activity is not equal to zero.

The protein concentration can be determined using known methods, for example the BCA method (bicinchoninic acid; 2,2′-bichinolyl-4,4′-dicarboxylic acid) or the Biuret method (Gornall et al., 1948, J. Biol. Chem., 177:751-766). The active protein concentration can be determined in this regard by titrating the active centers using a suitable irreversible inhibitor and determining the residual activity (Bender et al., 1966, J. Am. Chem. Soc. 88(24): 5890-5913).

All conceivable types of washing or cleaning agents are to be understood as washing or cleaning agents, both concentrates and undiluted agents, for use on a commercial scale, in washing machines or for hand washing or cleaning. These include, for example, washing agents for textiles, carpets or natural fibers, for which the term washing agent is used. These also include, for example, dishwashing detergents for dishwashers (automatic dishwashing detergents) or manual dishwashing detergents or cleaners for hard surfaces such as metal, glass, porcelain, ceramics, tiles, stone, painted surfaces, plastics, wood or leather, for which the term cleaning agent is used, i.e., in addition to manual and automatic dishwashing detergents, also, for example, scouring agents, glass cleaners, WC rim blocks, etc. The washing and cleaning agents also include auxiliary washing agents which are added to the actual washing agent during manual or automatic textile washing in order to achieve a further effect. Furthermore, washing and cleaning agents also include textile pre-treatment and post-treatment agents, i.e., the agents with which the item of laundry is brought into contact before the actual washing cycle, for example to loosen stubborn stains, and also the agents which give the laundry further desirable properties such as a pleasant feel, crease resistance or low static charge in a step subsequent to the actual textile wash. The agents mentioned last include fabric softeners, inter alia.

The dishwashing detergent can be an automatic dishwashing detergent but also a manual dishwashing detergent. Automatic dishwashing detergents are cleaning agents that have been optimized for use in automatic dishwashers. Manual dishwashing detergents are optimized for hand washing. The agents are preferably automatic dishwashing detergents. The agents are particularly preferably liquid automatic dishwashing detergents.

The washing or cleaning agents, which may be in the form of powdered solids, in further-compacted particulate form, as homogeneous solutions or suspensions, may contain, in addition to a protease and a stabilizer compound, all known ingredients conventional in such agents, with preferably at least one other ingredient being present in the agent. The agents can in particular contain surfactants, builders, polymers, glass corrosion inhibitors, corrosion inhibitors, bleaching agents such as peroxygen compounds, bleach activators or bleach catalysts. They may also contain water-miscible organic solvents, further enzymes, enzyme stabilizers, sequestering agents, electrolytes, pH regulators and/or further auxiliaries such as optical brighteners, graying inhibitors, dye transfer inhibitors, foam regulators, as well as dyes and fragrances, and combinations thereof.

The non-ionic surfactants are a preferred component of the washing and cleaning agents, wherein non-ionic surfactants of the general formula R¹—CH(OH)CH₂O-(AO)_{w}-(A′O)ₓ-(A″O)_{y}-(A″′O)_{z}—R² are preferred, where R¹ represents a straight-chain or branched, saturated or mono- or polyunsaturated C₆₋₂₄-alkyl or -alkenyl functional group; R² represents a linear or branched hydrocarbon functional group having 2 to 26 carbon atoms; A, A′, A′″ and A″″ represent, independently of one another, a functional group from the group —CH₂CH₂, —CH₂CH₂—CH₂, —CH₂—CH(CH₃), —CH₂—CH₂—CH₂—CH₂, —CH₂—CH(CH₃)—CH₂—, —CH₂—CH(CH₂—CH₃), and w, x, y and z represent values between 0.5 and 120, where x, y and/or z can also be 0.

By adding the above-mentioned non-ionic surfactants of the general formula R¹—CH(OH)CH₂O-(AO)_{w}-(A′O)ₓ-(A″O)_{y}-(A″′O)_{z}—R², subsequently also referred to as “hydroxy mixed ethers,” the cleaning performance of enzyme-containing preparations can surprisingly be significantly improved, both in comparison with surfactant-free systems and in comparison with systems containing alternative non-ionic surfactants, for example from the group of polyalkoxylated fatty alcohols.

By using these non-ionic surfactants having one or more free hydroxyl groups on one or both terminal alkyl functional groups, the stability of the enzymes contained in the washing or cleaning agent preparations can be improved substantially.

Particularly preferred are end-capped poly(oxyalkylated) non-ionic surfactants which, according to the formula R¹O[CH₂CH₂O]ₓCH₂CH(OH)R², also comprise, in addition to a functional group R¹, which represents linear or branched, saturated or unsaturated, aliphatic or aromatic hydrocarbon functional groups having 2 to 30 carbon atoms, preferably having 4 to 22 carbon atoms, a linear or branched, saturated or unsaturated, aliphatic or aromatic hydrocarbon functional group R² having 1 to 30 carbon atoms, where x represents values between 1 and 90, preferably values between 30 and 80, and in particular values between 30 and 60.

Surfactants of the formula R¹O[CH₂CH(CH₃)O]ₓ[CH₂CH₂O]_{y}CH₂CH(OH)R² are particularly preferred, where R¹ represents a linear or branched aliphatic hydrocarbon functional group having 4 to 18 carbon atoms or mixtures thereof, R² represents a linear or branched hydrocarbon functional group having 2 to 26 carbon atoms or mixtures thereof, and x represents values between 0.5 and 1.5, and y represents a value of at least 15. The group of these non-ionic surfactants includes, for example, C₂₋₂₆ fatty alcohol (PO)₁-(EO)₁₅₋₄₀-2-hydroxyalkyl ethers, in particular including C₈₋₁₀ fatty alcohol (PO)₁-(EO)₂₂-2-hydroxydecyl ethers.

Particularly preferred are also end-capped poly(oxyalkylated) non-ionic surfactants of the formula R¹O[CH₂CH₂O]ₓ[CH₂CH(R³)O]_{y}CH₂CH(OH)R², where R¹ and R² represent, independently of one another, a linear or branched, saturated or mono- or polyunsaturated hydrocarbon functional group having 2 to 26 carbon atoms, R³ is selected, independently of one another, from —CH₃, —CH₂CH₃, —CH₂CH₂—CH₃, —CH(CH₃)₂, but preferably represents —CH₃, and x and y represent, independently of one another, values between 1 and 32, non-ionic surfactants having R³=—CH₃ and values for x of from 15 to 32 and for y of 0.5 and 1.5 being very particularly preferred.

Further non-ionic surfactants that can preferably be used are the end-capped poly(oxyalkylated) non-ionic surfactants of the formula R¹O[CH₂CH(R₃)O]ₓ[CH₂]ₖCH(OH)[CH₂]ⱼOR², where R¹ and R² represent linear or branched, saturated or unsaturated, aliphatic or aromatic hydrocarbon functional groups having 1 to 30 carbon atoms, R³ represents H or a methyl, ethyl, n-propyl, iso-propyl, n-butyl, 2-butyl or 2-methyl-2-butyl functional group, x represents values between 1 and 30, and k and j represent values between 1 and 12, preferably between 1 and 5. If the value x is ≥2, each R³ in the above formula R¹O[CH₂CH(R³)O]ₓ[CH₂]ₖCH(OH)[CH₂]ⱼOR² can be different. R¹ and R² are preferably linear or branched, saturated or unsaturated, aliphatic or aromatic hydrocarbon functional groups having 6 to 22 carbon atoms, with functional groups having 8 to 18 C atoms being particularly preferred. For the functional group R³, H, —CH₃ or —CH₂CH₃ are particularly preferred. Particularly preferred values for x are in the range from 1 to 20, in particular from 6 to 15.

As described above, each R³ in the above formula can be different if x is ≥2. In this way, the alkylene oxide unit in square brackets can be varied. For example, if x represents 3, the functional group R³ can be selected in order to form ethylene oxide (R³=H) or propylene oxide (R³=CH³) units, which can be joined together in any sequence, for example (EO)(PO)(EO), (EO)(EO)(PO), (EO)(EO)(EO), (PO)(EO)(PO), (PO)(PO)(EO), and (PO)(PO)(PO). The value 3 for x has been selected here as an example and can by all means be greater, wherein the range of variation increases as the values for x increase and includes a large number of (EO) groups combined with a small number of (PO) groups, or vice versa.

Particularly preferred end-capped poly(oxyalkylated) alcohols of the above formula have values of k=1 and j=1, and therefore the previous formula is simplified to R¹O[CH₂CH(R³)O]ₓCH₂CH(OH)CH₂OR². In the formula mentioned last, R¹, R² and R³ are as defined above and x represents numbers from 1 to 30, preferably from 1 to 20, and in particular from 6 to 18. Surfactants, in which the functional groups R¹ and R² have 9 to 14 C atoms, R³ represents H, and x assumes values from 6 to 15, are particularly preferred.

Finally, the non-ionic surfactants of the general formula R¹—CH(OH)CH₂O-(AO)_{w}—R² have proven to be particularly effective, where R¹ represents a straight-chain or branched, saturated or mono- or polyunsaturated C₆₋₂₄-alkyl or -alkenyl functional group; R² represents a linear or branched hydrocarbon functional group having 2 to 26 carbon atoms; A represents a functional group from the group —CH₂CH₂, —CH₂CH₂—CH₂, —CH₂—CH(CH₃), and w represents values between 1 and 120, preferably 10 to 80, in particular 20 to 40. The group of these non-ionic surfactants includes, for example, C₄₋₂₂ fatty alcohol-(EO)₁₀₋₈₀-2-hydroxyalkyl ethers, in particular also C₈₋₁₂ fatty alcohol-(EO)₂₂-2-hydroxydecyl ethers and C₄₋₂₂ fatty alcohol-(EO)₄₀₋₈₀-2-hydroxyalkyl ethers.

Preferred washing and cleaning agents are characterized in that the washing and cleaning agent contains at least one non-ionic surfactant, preferably a non-ionic surfactant from the group of hydroxy mixed ethers, the percentage by weight of the non-ionic surfactant with respect to the total weight of the washing and cleaning agent preferably being from 0.2 to 10 wt. %, more preferably from 0.4 to 7.0 wt. % and in particular from 0.6 to 6.0 wt. %.

Preferred agents for use in automatic dishwashing methods can contain, in addition to the non-ionic surfactants described above, further surfactants, in particular amphoteric surfactants. However, the proportion of anionic surfactants with respect to the total weight of these agents is preferably limited. Preferred automatic dishwashing detergents are therefore characterized in that they contain less than 5.0 wt. %, preferably less than 3.0 wt. %, particularly preferably less than 2.0 wt. %, of anionic surfactant, based on the total weight thereof. Larger quantities of anionic surfactants are not used, in particular so as to avoid excessive foaming.

Another preferred component of agents are complexing agents. Particularly preferred complexing agents are the phosphonates, provided that their use is permitted by regulations. In addition to 1-hydroxyethane-1,1-diphosphonic acid, the complexing phosphonates include a number of different compounds such as diethylenetriamine penta(methylene phosphonic acid) (DTPMP). Hydroxy alkane or amino alkane phosphonates are particularly preferred in this application. Among the hydroxy alkane phosphonates, 1-hydroxyethane-1,1-diphosphonate (HEDP) has particular significance as a cobuilder. It is preferably used as a sodium salt, the disodium salt reacting neutral and the tetrasodium salt reacting alkaline (pH 9). Possible aminoalkane phosphonates preferably include ethylenediamine tetramethylene phosphonate (EDTMP), diethylentriamine pentamethylene phosphonate (DTPMP) and the higher homologs thereof. They are preferably used in the form of the neutral-reacting sodium salt, for example as the hexasodium salt of EDTMP or as the heptasodium and octasodium salt of DTPMP. Of the class of phosphonates, HEDP is preferably used as a builder. The aminoalkane phosphonates additionally have a pronounced heavy-metal-binding power. Accordingly, it may be preferred, in particular if the agents also contain bleach, to use aminoalkane phosphonates, in particular DTPMP, or to use mixtures of the mentioned phosphonates.

A preferred agent in the context of this application contains one or more phosphonate(s) from the group aminotrimethylene phosphonic acid (ATMP) and/or the salts thereof; ethylenediamine tetra(methylene phosphonic acid) (EDTMP) and/or the salts thereof; diethylenetriamine penta(methylene phosphonic acid) (DTPMP) and/or the salts thereof; 1-hydroxyethane-1,1-diphosphonic acid (HEDP) and/or the salts thereof; 2-phosphonobutane-1,2,4-tricarboxylic acid (PBTC) and/or the salts thereof; hexamethylenediamine tetra(methylene phosphonic acid) (HDTMP) and/or the salts thereof; and nitrilotri(methylenephosphonic acid) (NTMP) and/or the salts thereof.

Particularly preferred agents are those which contain 1-hydroxyethane-1,1-diphosphonic acid (HEDP) or diethylenetriamine penta(methylene phosphonic acid) (DTPMP) as phosphonates. The agent may, of course, contain two or more different phosphonates. Agents that are preferred are characterized in that the agent contains at least one complexing agent from the group of phosphonates, preferably 1-hydroxyethane-1,1-diphosphonate, the proportion by weight of the phosphonate with respect to the total weight of the cleaning agent preferably being between 0.1 and 8.0 wt. %, more preferably 0.2 and 5.0 wt. % and in particular 0.5 and 3.0 wt. %.

The agents also preferably contain builder. The builders include in particular silicates, carbonates and organic cobuilders.

Polycarboxylates/polycarboxylic acids, polymeric polycarboxylates, aspartic acid, polyacetals, dextrins, other organic cobuilders and phosphonates are particularly noteworthy as organic cobuilders. These classes of substances are described below. Organic cobuilder substances can, if desired, be contained in amounts of up to 40 wt. %, in particular up to 25 wt. %, and preferably from 1 to 8 wt. %.

Usable organic builder substance are, for example, the polycarboxylic acids that can be used in the form of the free acids and/or the sodium salts thereof, where polycarboxylic acids are understood to mean the carboxylic acids which carry more than one acid function. For example, these are citric acid, adipic acid, succinic acid, glutaric acid, malic acid, tartaric acid, maleic acid, fumaric acid, saccharic acid, carboxylmethylinulin, monomeric and polymeric aminopolycarboxylic acids, in particular glycinediacetic acid, methylglycinediacetic acid, nitrilotriacetic acid (NTA), iminodisuccinates such as ethylenediamine-N,N′-disuccinic acid and hydroxyiminodisuccinate, ethylenediaminetetraacetic acid and polyaspartic acid, polyphosphonic acids, in particular aminotris(methylenephosphonic acid), ethylenediamine tetrakis(methylenephosphonic acid), lysine tetra(methylenephosphonic acid) and 1-hydroxyethane-1,1-diphosphonic acid, polymeric hydroxy compounds such as dextrin, and polymeric (poly)carboxylic acids, polycarboxylates which can be obtained in particular by oxidizing polysaccharides or dextrins, and/or polymeric acrylic acids, methacrylic acids, maleic acids, and mixed polymers thereof, which may also contain, polymerized in the polymer, small proportions of polymerizable substances, without a carboxylic acid functionality. Organic builder substances of this kind can, if desired, be contained in amounts of up to 50 wt. %, in particular up to 25 wt. %, and preferably from 10 to 20 wt. %.

In addition to their builder effect, the free acids typically also have the property of being an acidification component and are thus also used for setting a lower and milder pH of washing or cleaning agents. Particularly noteworthy here are citric acid, succinic acid, glutaric acid, adipic acid, gluconic acid, and any mixtures thereof. Citric acid or salts of citric acid are particularly preferably used as the builder. Further particularly preferred builder substances are selected from methylglycine diacetic acid (MGDA), glutamine diacetic acid (GLDA), aspartic acid diacetate (ASDA), hydroxyethyl-iminodiacetate (HEIDA), iminodisuccinate (IDS), ethylenediamine disuccinate (EDDS), carboxymethyl inulin and polyaspartate.

In preferred embodiments, citric acid and/or citrate is used as the water-soluble, organic builder. It is particularly preferred to use 5 to 25 wt. %, preferably 7.5 to 12.5 wt. %, citric acid and/or 5 to 25 wt. %, preferably 7.5 to 12.5 wt. %, citrate, preferably alkali citrate, more preferably sodium citrate. Citric acid/citrate can each be used in the form of their hydrates, for example citric acid can be used in the form of the monohydrate, and citrate can be used in the form of the trisodium citrate dihydrate.

Polymeric polycarboxylates are also suitable as builders. These are, for example, the alkali metal salts of polyacrylic acid or polymethacrylic acid, for example those having a relative molecular mass of from 500 to 70,000 g/mol. For the purpose of this document, the molar masses indicated for polymeric polycarboxylates are weight-average molar masses M_{w} of the particular acid form which have been determined in principle using gel permeation chromatography (GPC), a UV detector having been used. The measurement was carried out against an external polyacrylic acid standard which, owing to the structural relationship thereof with the tested polymers, yields realistic molecular weight values. These specifications differ significantly from the molecular weight specifications for which polystyrene sulfonic acids are used as the standard. The molar masses measured against polystyrene sulfonic acids are generally considerably higher than the molar masses indicated in the present application.

Suitable polymers are in particular polyacrylates which preferably have a molecular mass of from 2,000 to 20,000 g/mol. Due to their superior solubility, the short-chain polyacrylates, which have molar masses of from 2,000 to 10,000 g/mol, and particularly preferably from 3,000 to 5,000 g/mol, can in turn be preferred from this group.

In addition, copolymeric polycarboxylates are suitable, in particular those of acrylic acid with methacrylic acid and of acrylic acid or methacrylic acid with maleic acid. Copolymers of acrylic acid with maleic acid which contain from 50 to 90 wt. % of acrylic acid and from 50 to 10 wt. % of maleic acid have been found to be particularly suitable. The relative molecular mass thereof, based on free acids, is generally from 2,000 to 70,000 g/mol, preferably from 20,000 to 50,000 g/mol, and in particular from 30,000 to 40,000 g/mol.

In addition to the above-described builders, polymers having a cleaning action can also be present in the washing or cleaning agent. The proportion by weight of the polymers having a cleaning action with respect to the total weight of washing or cleaning agent is preferably from 0.1 to 20 wt. %, more preferably from 1.0 to 15 wt. % and in particular from 2.0 to 12 wt. %.

Polymers containing sulfonic acid groups, in particular from the group of copolymeric polysulfonates, are preferably used as polymers having a cleaning action. These copolymeric polysulfonates contain, in addition to sulfonic acid group-containing monomer(s), at least one monomer from the group of unsaturated carboxylic acids.

As unsaturated carboxylic acid(s), unsaturated carboxylic acids of formula R¹(R²)C═C(R³)COOH are particularly preferably used, where R¹ to R³ represent, independently of one another, —H, —CH₃, a straight-chain or branched saturated alkyl functional group having 2 to 12 carbon atoms, a straight-chain or branched, mono- or polyunsaturated alkenyl functional group having 2 to 12 carbon atoms, —NH₂, —OH, or —COOH-substituted alkyl or alkenyl functional groups as defined above, or represent —COOH or —COOR⁴, where R⁴ is a saturated or unsaturated, straight-chain or branched hydrocarbon functional group having 1 to 12 carbon atoms. Particularly preferred unsaturated carboxylic acids are acrylic acid, methacrylic acid, ethacrylic acid, α-chloroacrylic acid, α-cyanoacrylic acid, crotonic acid, α-phenylacrylic acid, maleic acid, maleic anhydride, fumaric acid, itaconic acid, citraconic acid, methylene malonic acid, sorbic acid, cinnamic acid, or mixtures thereof. Unsaturated dicarboxylic acids can of course also be used.

For sulfonic acid group-containing monomers, those of the formula R⁵(R⁶)C═C(R⁷)—X—SO₃H are preferred, where R⁵ to R⁷, independently of one another, represent —H, —CH₃, a straight-chain or branched saturated alkyl functional group having 2 to 12 carbon atoms, a straight-chain or branched, mono- or polyunsaturated alkenyl functional group having 2 to 12 carbon atoms, —NH₂, —OH, or —COOH-substituted alkyl or alkenyl functional groups, or represent —COOH or —COOR⁴, where R⁴ is a saturated or unsaturated, straight-chain or branched hydrocarbon functional group having 1 to 12 carbon atoms, and X represents an optionally present spacer group that is selected from —(CH₂)ₙ—, where n=0 to 4, —COO—(CH₂)ₖ—, where k=1 to 6, —C(O)—NH—C(CH₃)₂—, —C(O)—NH—C(CH₃)₂—CH₂— and —C(O)—NH—CH(CH₂CH₃)—.

Among these monomers, those of formulas H₂C═CH—X—SO₃H, H₂C═C(CH₃)—X—SO₃H and HO₃S—X—(R⁶)C═C(R⁷)—X—SO₃H are preferred, where R⁶ and R⁷, independently of one another, are selected from —H, —CH₃, —CH₂CH₃, —CH₂CH₂CH₃ and —CH(CH₃)₂, and X represents an optionally present spacer group that is selected from —(CH₂)ₙ—, where n=0 to 4, —COO—(CH₂)ₖ—, where k=1 to 6, —C(O)—NH—C(CH₃)₂—, —C(O)—NH—C(CH₃)₂—CH₂— and —C(O)—NH—CH(CH₂CH₃)—.

Particularly preferred sulfonic acid group-containing monomers are 1-acrylamido-1-propanesulfonic acid, 2-acrylamido-2-propanesulfonic acid, 2-acrylamido-2-methyl-1-propanesulfonic acid, 2-methacrylamido-2-methyl-1-propanesulfonic acid, 3-methacrylamido-2-hydroxy-propanesulfonic acid, allyl sulfonic acid, methallyl sulfonic acid, allyloxybenzene sulfonic acid, methallyloxybenzene sulfonic acid, 2-hydroxy-3-(2-propenyl oxy)propanesulfonic acid, 2-methyl-2-propene-1-sulfonic acid, styrene sulfonic acid, vinyl sulfonic acid, 3-sulfopropyl acrylate, 3-sulfopropyl methacrylate, sulfomethacrylamide, sulfomethylmethacrylamide, as well as mixtures of the above acids or water-soluble salts thereof.

In the polymers, the sulfonic acid groups can be present entirely or partially in neutralized form. The use of partially or fully neutralized sulfonic acid group-containing copolymers is preferred. The molar mass of the sulfo-copolymers that are preferably used can be varied in order to adapt the properties of the polymers to the desired intended use. Preferred automatic dishwashing agents are characterized in that the copolymers have molar masses of 2,000 to 200,000 g/mol, preferably 4,000 to 25,000 g/mol and in particular 5,000 to 15,000 g/mol.

In another preferred embodiment, the copolymers comprise not only carboxyl group-containing monomers and sulfonic acid group-containing monomers but also at least one non-ionic, preferably hydrophobic monomer. Through the use of these hydrophobically modified polymers, it was possible to improve, in particular, the rinsing performance of automatic dishwashing detergents.

Washing and cleaning agents containing a copolymer comprising i) carboxylic acid group-containing monomer(s), ii) sulfonic acid group-containing monomer(s), or iii) non-ionic monomer(s) are preferred. Through the use of these terpolymers, it was possible to improve the rinsing performance of automatic dishwashing detergents over comparable dishwashing detergents comprising sulfo-polymers without the addition of non-ionic monomers.

As the non-ionic monomers, monomers of the general formula R¹(R²)C═C(R³)—X—R⁴ are preferably used, where R¹ to R³ represent, independently of one another, —H, —CH₃ or —C₂H₅, X represents an optionally present spacer group selected from —CH₂—, —C(O)O— and —C(O)—NH—, and R⁴ represents a straight-chain or branched saturated alkyl functional group having 2 to 22 carbon atoms or an unsaturated, preferably aromatic, functional group having 6 to 22 carbon atoms. Particularly preferred non-ionic monomers are butene, isobutene, pentene, 3-methylbutene, 2-methylbutene, cyclopentene, hexene, hexene-1, 2-methlypentene-1, 3-methlypentene-1, cyclohexene, methylcyclopentene, cycloheptene, methylcyclohexene, 2,4,4-trimethylpentene-1, 2,4,4-trimethylpentene-2, 2,3-dimethylhexene-1, 2,4-dimethylhexene-1, 2,5-dimethylhexene-1, 3,5-dimethylhexene-1,4,4-dimethylhexane-1, ethylcyclohexene, 1-octene, α-olefins having 10 or more carbon atoms such as 1-decene, 1-dodecene, 1-hexadecene, 1-octadecene and C₂₂-α-olefin, 2-styrene, α-methyl styrene, 3-methyl styrene, 4-propyl styrene, 4-cyclohexylstyrene, 4-dodecyl styrene, 2-ethyl-4-benzylstyrene, 1-vinyl naphthalene, 2-vinyl naphthalene, acrylic acid methyl ester, acrylic acid ethyl ester, acrylic acid propyl ester, acrylic acid butyl ester, acrylic acid pentyl ester, acrylic acid hexyl ester, methacrylic acid methyl ester, N-(methyl)acrylamide, acrylic acid-2-ethylhexyl ester, methacrylic acid-2-ethylhexyl ester, N-(2-ethylhexyl)acrylamide, acrylic acid octyl ester, methacrylic acid octyl ester, N-(octyl)acrylamide, acrylic acid lauryl ester, methacrylic acid lauryl ester, N-(lauryl)acrylamide, acrylic acid stearyl ester, methacrylic acid stearyl ester, N-(stearyl)acrylamide, acrylic acid behenyl ester, methacrylic acid behenyl ester and N-(behenyl)acrylamide, or mixtures thereof.

The proportion by weight of the sulfonic acid group-containing copolymers with respect to the total weight of agents is preferably from 0.1 to 15 wt. %, more preferably from 1.0 to 12 wt. % and in particular from 2.0 to 10 wt. %.

Possible peroxygen compounds suitable for use in the agents include, in particular, organic peroxy acids or peracid salts of organic acids, such as phthalimidopercaproic acid, perbenzoic acid, or salts of diperdodecanoic diacid, hydrogen peroxide and inorganic salts giving off hydrogen peroxide under the washing conditions, which salts include perborate, percarbonate, persilicate, and/or persulfates such as caroate, as well as hydrogen peroxide inclusion compounds such as H₂O₂-urea adducts. Hydrogen peroxide can also be produced by means of an enzymatic system, i.e., an oxidase and the substrate thereof. If solid peroxygen compounds are intended to be used, these may be used in the form of powders or granules, which may also be coated in a manner known in principle. The peroxygen compounds can be added to the washing liquor as such or in the form of the agents containing them, which in principle can contain all conventional washing, cleaning or disinfectant components. Particularly preferably, alkali percarbonate, or alkali perborate monohydrate is used. If an agent contains peroxygen compounds, these are present in amounts of preferably up to 50 wt. %, in particular from 5 to 30 wt. %, more preferably from 0.1 to 20 wt. %.

Compounds which, under perhydrolysis conditions, result in aliphatic peroxocarboxylic acids having preferably 1 to 10 C atoms, in particular 2 to 4 C atoms, and/or optionally substituted perbenzoic acid, may be used in the agents as bleach activators. Substances that carry the O-acyl and/or N-acyl groups of the stated number of C atoms and/or optionally substituted benzoyl groups are suitable. Preferred are polyacylated alkylene diamines, in particular tetraacetylethylenediamine (TAED), acylated triazine derivatives, in particular 1,5-diacetyl-2,4-dioxohexahydro-1,3,5-triazine (DADHT), acylated glycolurils, in particular tetraacetylglycoluril (TAGU), N-acylimides, in particular N-nonanoylsuccinimide (NOSI), acylated phenol sulfonates or carboxylates or the sulfonic or carboxylic acids thereof, in particular nonanoyloxybenzenesulfonate or isononanoyloxybenzenesulfonate or laroyloxybenzenesulfonate (NOBS or iso-NOBS or LOBS), 4-(2-decanoyloxyethoxycarbonyloxy)-benzenesulfonate (DECOBS) or decanoyloxybenzoate (DOBA), carboxylic acid anhydrides, in particular phthalic acid anhydride, acylated polyhydric alcohols, in particular triacetin, ethylene glycol diacetate, 2,5-diacetoxy-2,5-dihydrofuran and enol esters, as well as acetylated sorbitol and mannitol or the described mixtures thereof (SORMAN), acylated sugar derivatives, in particular pentaacetyl glucose (PAG), pentaacetyl fructose, tetraacetylxylose and octaacetyl lactose, acetylated, optionally N-alkylated glucamine and gluconolactone, N-acylated lactams, for example N-benzoylcaprolactam, nitriles from which perimidic acids are formed, in particular aminoacetonitrile derivatives having a quaternized nitrogen atom, and/or oxygen-transferring sulfonimines and/or acylhydrazones. The hydrophilically substituted acyl acetals and the acyl lactams are likewise preferably used. Combinations of conventional bleach activators can also be used. Such bleach activators can, in particular in the presence of the above-mentioned hydrogen peroxide-yielding bleaching agents, be present in the customary quantity range, preferably in amounts of from 0.5 to 10 wt. %, and in particular 1 to 8 wt. %, based on the total agent, but are preferably entirely absent when percarboxylic acid is used as the sole bleaching agent.

In addition to or instead of the conventional bleach activators, sulfonimines and/or bleach-boosting transition metal salts or transition metal complexes may also be contained in solid agents as what are referred to as bleach catalysts.

A dishwashing detergent also comprises a bleach activator. These substances are preferably bleach-intensifying transition metal salts or transition metal complexes such as Mn, Fe, Co, Ru or Mo salen complexes or carbonyl complexes. Mn, Fe, Co, Ru, Mo, Ti, V, and Cu complexes having N-containing tripod ligands as well as Co, Fe, Cu, and Ru ammine complexes can also be used as bleach catalysts.

Complexes of manganese in oxidation stage II, III, IV, or IV are particularly preferably used which preferably contain one or more macrocyclic ligands with the donor functions N, NR, PR, O and/or S. Preferably, ligands are used which have nitrogen donor functions. It is particularly preferred to use bleach catalyst(s) in the agents which contain, as macromolecular ligands, 1,4,7-trimethyl-1,4,7-triazacyclononane (Me-TACN), 1,4,7-triazacyclononane (TACN), 1,5,9-trimethyl-1,5,9-triazacyclododecane (Me-TACD), 2-methyl-1,4,7-trimethyl-1,4,7-triazacyclononane (Me/Me-TACN), and/or 2-methyl-1,4,7-triazacyclononane (Me/TACN). Suitable manganese complexes are, for example, [Mn^{III}₂(μ-O)₁(μ-OAc)₂(TACN)₂](CIO₄)₂, [Mn^{III}Mn^{IV}(μ-O)₂(μ-OAc)₁(TACN)₂](BPh₄)₂, [Mn^{IV}₄(μ-O)₆(TACN)₄](CIO₄)₄, [Mn^{III}₂(μ-O)₁(μ-OAc)₂(Me-TACN)₂](CIO₄)₂, [Mn^{III}Mn^{IV}(μ-O)₁(μ-OAc)₂(Me-TACN)₂](CIO₄)₃, [Mn^{IV}2(μ-O)₃(Me-TACN)₂](PF₆)₂ and [Mn^{IV}₂(μ-O)₃(Me/Me-TACN)₂](PF₆)₂(OAc═OC(O)CH₃).

Dishwashing detergents, in particular automatic dishwashing detergents, characterized in that they contain a bleach catalyst selected from the group of the bleach-intensifying transition metal salts and transition metal complexes, preferably from the group of the complexes of manganese with 1,4,7-trimethyl-1,4,7-triazacyclononane (Me-TACN) or 1,2,4,7-tetramethyl-1,4,7-triazacyclononane (Me/Me-TACN) are preferred, since the aforementioned bleach catalysts in particular can significantly improve the cleaning result.

The aforementioned bleach-intensifying transition metal complexes, in particular having the central atoms Mn and Co, are preferably used in an amount of up to 5 wt. %, in particular 0.0025 to 1 wt. % and particularly preferably 0.01 to 0.30 wt. %, in each case based on the total weight of the bleach catalyst-containing agents. In special cases, however, more bleach catalyst can also be used.

The agents can contain an organic solvent as a further component. Adding organic solvents has an advantageous effect on the enzyme stability and cleaning performance of these agents. Preferred organic solvents are derived from the group of monohydric or polyhydric alcohols, alkanolamines or glycol ethers. The solvents are preferably selected from ethanol, n-propanol or i-propanol, butanol, glycol, propanediol or butanediol, glycerol, diglycol, propyl diglycol or butyl diglycol, hexylene glycol, ethylene glycol methyl ether, ethylene glycol ethyl ether, ethylene glycol propyl ether, ethylene glycol mono-n-butyl ether, diethylene glycol methyl ether, diethylene glycol ethyl ether, propylene glycol methyl ether, propylene glycol ethyl ether or propylene glycol propyl ether, dipropylene glycol methyl ether or dipropylene glycol ethyl ether, methoxytriglycol, ethoxytriglycol or butoxytriglycol, 1-butoxyethoxy-2-propanol, 3-methyl-3-methoxybutanol, propyl ene-glycol-t-butyl ether, and mixtures of these solvents. The proportion by weight of these organic solvents with respect to the total weight of the agents is preferably from 0.1 to 10 wt. %, more preferably from 0.2 to 8.0 wt. % and in particular from 0.5 to 5.0 wt. %. A particularly preferred organic solvent which is particularly effective in stabilizing the cleaning agents is glycerol, as well as 1,2-propylene glycol. Liquid agents which contain at least one polyol, preferably from the group of glycerol and 1,2-propylene glycol, are preferred, the proportion by weight of the polyol with respect to the total weight of the cleaning agent preferably being from 0.1 to 10 wt. %, more preferably from 0.2 to 8.0 wt. %, and in particular from 0.5 to 5.0 wt. %. Other preferred organic solvents are the organic amines and alkanolamines. The agents preferably contain these amines in amounts of from 0.1 to 10 wt. %, preferably from 0.2 to 8.0 wt. % and in particular from 0.5 to 5.0 wt. %, in each case based on the total weight thereof. Ethanolamine is a particularly preferred alkanolamine.

A further preferred component of the washing and cleaning agents is a sugar alcohol (alditol). The group of alditols includes non-cyclic polyols of the formula HOCH₂[CH(OH)]ₙCH₂OH. The alditols include, e.g., mannitol, isomalt, lactitol, sorbitol and xylitol, threitol, erythritol and arabitol. Sorbitol has been found to be particularly advantageous with regard to enzyme stability. The percentage by weight of the sugar alcohol with respect to the total weight of the washing and cleaning agent is preferably from 1.0 to 10 wt. %, more preferably from 2.0 to 8.0 wt. % and in particular from 3.0 to 6.0 wt. %.

An agent advantageously contains the protease in an amount of from 2 μg to 20 mg, preferably from 5 μg to 17.5 mg, particularly preferably from 20 μg to 15 mg and most particularly preferably from 50 μg to 10 mg per g of the agent. Further, the protease contained in the agent, and/or other ingredients of the agent, may be coated with a substance which is impermeable to the enzyme at room temperature or in the absence of water, and which becomes permeable to the enzyme under conditions of use of the agent. Such an embodiment is thus characterized in that the protease is coated with a substance which is impermeable to the protease at room temperature or in the absence of water. Furthermore, the washing or cleaning agent itself may also be packaged in a container, preferably an air-permeable container, from which it is released shortly before use or during the washing or rinsing process.

These embodiments include all solid, powdered, granular, tablet-form, liquid, gel or pasty administration forms of agents, which may optionally also consist of a plurality of phases and can be present in compressed or uncompressed form. The agent may be present as a flowable powder, in particular having a bulk density of from 300 to 1,200 g/L, in particular from 500 to 900 g/L or from 600 to 850 g/L. The solid administration forms of the agent also include extrudates, granules, tablets or pouches containing solid agents, which can be present both in large containers and in portions. Alternatively, the agent may also be in liquid, gel or pasty form, for example in the form of a non-aqueous agent or a non-aqueous paste or in the form of an aqueous agent or a water-containing paste. The agent may also be present as a single-component system. Such agents consist of one phase. Alternatively, an agent may also consist of a plurality of phases (multi-component system). Such an agent is accordingly divided into a plurality of components, for example two liquid, two solid or one liquid and one solid phase. The water-based and/or organic solvent-based liquid product formats may be present in thickened form, namely in the form of gels.

A substance, e.g., a composition or an agent, is solid if it is in a solid physical state at 25° C. and 1,013 mbar.

A substance, e.g., a composition or an agent, is liquid if it is in the fluid physical state at 25° C. and 1,013 mbar. Liquid also includes gel form.

The agents are preferably present in liquid form. Preferred washing and cleaning agents contain more than 40 wt. %, preferably between 50 and 90 wt. % and in particular between 60 and 80 wt. %, water, based on the total weight thereof.

The cleaning agents described herein, in particular dishwashing agents, even more preferably automatic dishwashing agents, are preferably pre-packaged into dosing units. These dosing units preferably comprise the amount of active cleaning substances necessary for a cleaning cycle. Preferred dosing units have a weight of between 12 and 30 g, preferably between 14 and 26 g and in particular between 15 and 22 g. The volume of the aforementioned dosing units and the spatial shape thereof are particularly preferably selected so that the pre-packaged units can be dosed via the dosing chamber of a dishwasher. The volume of the metering unit is therefore preferably between 10 and 35 ml, preferably between 12 and 30 ml.

The agents, in particular automatic dishwashing detergents, in particular the prefabricated dosing units, particularly preferably have a water-soluble wrapping. The water-soluble wrapping is preferably made from a water-soluble film material, which is selected from the group consisting of polymers or polymer mixtures. The wrapping may be made up of one or of two or more layers of the water-soluble film material. The water-soluble film material of the first layer and of the additional layers, if present, may be the same or different. Particularly preferred are films which, e.g., can be glued and/or sealed to form packaging such as tubes or sachets after they have been filled with an agent.

The water-soluble packaging may have one or more chambers. The agent may be contained in one or more chambers, if present, of the water-soluble wrapping. The amount of agent preferably corresponds to the full or half dose required for a dishwashing cycle.

It is preferred that the water-soluble wrapping contains polyvinyl alcohol or a polyvinyl alcohol copolymer. Water-soluble wrappings containing polyvinyl alcohol or a polyvinyl alcohol copolymer exhibit good stability with a sufficiently high level of water solubility, in particular cold-water solubility. Suitable water-soluble films for producing the water-soluble wrapping are preferably based on a polyvinyl alcohol or a polyvinyl alcohol copolymer of which the molecular weight is in the range of from 5,000 to 1,000,000 g/mol, preferably from 20,000 to 500,000 g/mol, particularly preferably from 30,000 to 100,000 g/mol, and in particular from 40,000 to 80,000 g/mol. Suitable water-soluble films for use in the water-soluble wrappings of the water-soluble packaging are films which are sold by MonoSol LLC, for example under the names M8630, C8400 or M8900. Other suitable films include films named Solublon® PT, Solublon® GA, Solublon® KC or Solublon® KL from Aicello Chemical Europe GmbH or the films VF-HP from Kuraray.

Such water-soluble wrappings are also already described in patent applications WO2004031338A and WO2003099985A, the disclosure of which is hereby incorporated by reference in its entirety.

The enzymes are generally not provided in the form of pure protein, but rather in the form of stabilized, storable and transportable preparations. These pre-packaged preparations include, e.g., the solid preparations obtained through granulation, extrusion, or lyophilization or, in particular in the case of liquid or gel agents, solutions of the enzymes, which are advantageously maximally concentrated, have a low water content, and/or are supplemented with stabilizers or other auxiliaries. Moreover, it is possible to formulate two or more enzymes together such that a single granule exhibits a plurality of enzyme activities.

Washing or cleaning agents may exclusively contain a Bacillus gibsonii protease, as defined herein. Alternatively, they may also contain other enzymes in a concentration that is expedient for the effectiveness of the agent. A further embodiment is therefore represented by agents which further comprise one or more further enzymes. Further enzymes which can preferably be used are all enzymes which can exhibit catalytic activity in the agent, in particular a lipase, amylase, cellulase, hemicellulase, mannanase, tannase, xylanase, xanthanase, xytoglucanase, ß-glucosidase, pectinase, carrageenase, perhydrolase, oxidase, oxidoreductase or another protease, which is different from the proteases, as well as mixtures thereof. Enzymes are contained in the agent advantageously in an amount of from 1×10⁻⁸ to 5 wt. %, in each case based on active protein. Increasingly preferably, each further enzyme is contained in agents in an amount of from 1×10⁷ to 3 wt. %, from 0.00001 to 1 wt. %, from 0.00005 to 0.5 wt. %, from 0.0001 to 0.1 wt. % and particularly preferably from 0.0001 to 0.05 wt. %, based on active protein. Particularly preferably, the enzymes exhibit synergistic cleaning performance against specific stains or spots, i.e., the enzymes contained in the agent composition support one another in their cleaning performance. Very particularly preferably, there is such synergism between the protease contained and a further enzyme of an agent, including in particular between said protease and an amylase and/or a lipase and/or a mannanase and/or a cellulase and/or a pectinase. Synergistic effects may arise not only between different enzymes, but also between one or more enzymes and other ingredients of the agent.

Examples of proteases are the subtilisins BPN′ from Bacillus amyloliquefaciens and Carlsberg from Bacillus licheniformis, the protease PB92, the subtilisins 147 and 309, the protease from Bacillus lentus, subtilisin DY, and the enzymes thermitase, proteinase K and the proteases TW3 and TW7, which belong to the subtilases but no longer to the subtilisins in the narrower sense. Subtilisin Carlsberg is available in a further developed form under the trade name Alcalase® from Novozymes. The subtilisins 147 and 309 are sold by Novozymes under the trade names Esperase® and Savinase®, respectively. The protease variants marketed under the name BLAND are derived from the protease from Bacillus lentus DSM 5483. Other proteases that can be used are, for example, the enzymes available under the trade names Durazym®, Relase®, Everlase®, Nafizym®, Natalase®, Kannase® and Ovozyme® from Novozymes, the enzymes available under the trade names Purafect®, Purafect® OxP, Purafect® Prime, Excellase® and Properase® from Danisco/Genencor, the enzyme available under the trade name Protosol® from Advanced Biochemicals Ltd., the enzyme available under the trade name Wuxi® from Wuxi Snyder Bioproducts Ltd., the enzymes available under the trade names Proleather® and Protease P® from Amano Pharmaceuticals Ltd., and the enzyme available under the name Proteinase K-16 from Kao Corp. The proteases from Bacillus gibsonii and Bacillus pumilus, disclosed in international patent applications WO2008086916 and WO2007131656 are particularly preferably used. Further advantageously usable proteases are disclosed in patent applications WO9102792, WO2008007319, WO9318140, WO0144452, GB1243784, WO9634946, WO2002029024 and WO2003057246. Further proteases that can be used are those which are naturally present in the microorganisms Stenotrophomonas maltophilia, in particular Stenotrophomonas maltophilia K279a, Bacillus intermedius and Bacillus sphaericus.

Examples of amylases are α-amylases from Bacillus licheniformis, from Bacillus amyloliquefaciens or from Bacillus stearothermophilus, as well as in particular the developments thereof that have been improved for use in washing or cleaning agents. The enzyme from Bacillus licheniformis is available from Novozymes under the name Termamyl® and from Danisco/Genencor under the name Purastar® ST. Development products of this α-amylase are available from Novozymes under the trade names Duramyl® and Termamyl® ultra, from Danisco/Genencor under the name Purastar® OxAm, and from Daiwa Seiko Inc. as Keistase®. The α-amylase from Bacillus amyloliquefaciens is marketed by Novozymes under the name BAN®, and derived variants from the α-amylase from Bacillus stearothermophilus are marketed under the names BSG® and Novamyl®, also by Novozymes. Furthermore, the α-amylase from Bacillus sp. A 7-7 (DSM 12368) and cyclodextrin glucanotransferase (CGTase) from Bacillus agaradherens (DSM 9948) are particularly noteworthy for this purpose. Furthermore, the amylolytic enzymes can be used which are disclosed in international patent applications WO2003002711, WO2003054177 and WO2007079938, the disclosure of which is therefore expressly referred to or the disclosure of which is therefore expressly included in the present patent application. Fusion products of all mentioned molecules can also be used. Furthermore, the developments of the α-amylase from Aspergillus niger and A. oryzae, available under the trade name Fungamyl® from Novozymes, are suitable. Other commercial products that can advantageously be used are, for example, Amylase-LT®, and Stainzyme® or Stainzyme Ultra® or Stainzyme Plus®, as well as Amplify™ or Amplify Prime™, also from Novozymes. Variants of these enzymes that can be obtained by point mutations can also be used.

Examples of cellulases (endoglucanases, EG) include the fungal cellulase preparation which is rich in endoglucanase (EG) and the developments thereof which are provided by Novozymes under the trade name Celluzyme®. The products Endolase® and Carezyme®, also available from Novozymes, are based on 50 kD-EG and 43 kD-EG, respectively, from Humicola insolens DSM 1800. Further commercial products from this company that can be used are Cellusoft®, Renozyme®, and Celluclean®. It is also possible to use cellulases, for example, which are available from AB Enzymes under the trade names Ecostone® and Biotouch®, and which are, at least in part, based on 20 kD-EG from Melanocarpus. Other cellulases from AB Enzymes are Econase® and Ecopulp®. Other suitable cellulases are from Bacillus sp. CBS 670.93 and CBS 669.93, the cellulase from Bacillus sp. CBS 670.93 being available from Danisco/Genencor under the trade name Puradax®. Other commercial products that can be used from Danisco/Genencor are “Genencor detergent cellulase L” and IndiAge®Neutra.

Examples of lipases or cutinases, which are used in particular due to their triglyceride-cleaving activities, but also to generate peracids from suitable precursors in situ, include, for example, the lipases originally obtained from Humicola lanuginosa (Thermomyces lanuginosus) or further developed therefrom, in particular those with one or more of the following amino acid exchanges starting from the lipase mentioned in positions D96L, T213R and/or N233R, particularly preferably T213R and N233R. Lipases are sold, for example, by Novozymes under the trade names Lipolase®, Lipolase® Ultra, LipoPrime®, Lipozyme® and Lipex®. Another lipase that can be used advantageously is available from Novozymes under the trade name Lipoclean®. Moreover, the cutinases which have been originally isolated from Fusarium solani pisi and Humicola insolens can also be used, for example. Lipases that can also be used are available from Amano under the names Lipase CE®, Lipase P®, Lipase B®, and Lipase CES®, Lipase AKG®, Bacillus sp. Lipase®, Lipase AP®, Lipase M-AP® and Lipase AML®. From the company Danisco/Genencor, for example, lipases or cutinases can be used whose starting enzymes were originally isolated from Pseudomonas mendocina and Fusarium solanii. The preparations M1 Lipase® and Lipomax® originally marketed by Gist-Brocades (now Danisco/Genencor) and the enzymes marketed by Meito Sangyo KK under the names Lipase MY-30®, Lipase OF® and Lipase PL®, as well as the product Lumafast® from Danisco/Genencor should be mentioned as other important commercial products.

In order to increase the bleaching effect, oxidoreductases such as oxidases, oxygenases, catalases, peroxidases such as halo, chloro, bromo, lignin, glucose, or manganese peroxidases, dioxygenases or laccases (phenoloxidases, polyphenoloxidases) can be used. Advantageously, organic, particularly preferably aromatic compounds that interact with the enzymes are additionally added in order to potentiate the activity of the relevant oxidoreductases (enhancers) or, in the event of greatly differing redox potentials, to ensure the flow of electrons between the oxidizing enzymes and the contaminants (mediators).

A further objective is a method for cleaning textiles and/or hard surfaces, in particular dishes, characterized in that in at least one method step, an agent is used.

A preferred cleaning method is an automatic dishwashing method. The agent can be dosed into the cleaning liquor in such a method, for example by means of the dosing chamber in the door or by means of an additional dosing container in the interior of the dishwasher. Alternatively, the agent can also be applied directly to the dirty dishes or to one of the interior walls of the dishwasher, for example the inside of the door. The method is carried out in the interior of a commercially available dishwasher. In the case of a dishwasher, the cleaning program can generally be selected and determined by the user before the dishwashing method is carried out. The dishwasher cleaning program used in the method comprises at least one prewash cycle and one cleaning cycle. Cleaning programs which comprise further cleaning or rinsing cycles, for example a rinse cycle, are preferred. The method is particularly preferably part of a cleaning program comprising a prewash cycle, a cleaning cycle and a rinse cycle. The method is preferably used in connection with cleaning programs in which the washing liquor is heated during the cleaning cycle. In a preferred embodiment of the method, the cleaning cycle, during which the agent is dosed into the interior of the dishwasher, is characterized in that the temperature of the cleaning liquor during said cycle rises to values above 30° C., preferably above 40° C. and in particular above 50° C.

In various embodiments, the method described above is characterized in that the protease is used at a temperature of from 0 to 100° C., preferably 10 to 70° C., more preferably 30 to 50° C. and most preferably at 45° C.

Alternative embodiments of this subject matter are also represented by methods for cleaning textiles as well as methods for treating textile raw materials or for textile care, in which an agent is used in at least one method step. Among these, methods for textile raw materials, fibers or textiles comprising natural constituents are preferred, and very particularly for such materials, fibers or textiles comprising wool or silk.

Another objective is the use of a stabilizer compound selected from the group consisting of phenylboronic acid derivative, boric acid, peptide inhibitor, and combinations thereof for improving the stability of a Bacillus gibsonii protease in washing or cleaning agents and/or optionally further enzymes present in the washing or cleaning agent.

All aspects, subject matter, and embodiments described for the protease and agents containing same can also be applied to this subject matter. Therefore, reference is expressly made at this point to the disclosure at the appropriate point with the note that this disclosure also applies to the above-described use.

### Example

Storage stability of the enzymes in automatic dishwashing detergents

A commercially available liquid automatic dishwashing detergent was used. The dishwashing detergent matrix had the following composition:

| Raw material | % AM in formula |
|---|---|
| Water | to make up to 100% |
| CaCl₂ | 0.05-1% |
| Na citrate | 3-20% |
| Citric acid | 0.5-3% |
| Sulfo-polymer (Acusol 590) | 3-7% |
| Thickening agent | 0-0.9% |
| (xanthan gum) | |
| MGDA | 12-25% |
| Preservatives | <1.5% |
| Non-ionic surfactant | 1-4% |
| Hydrophilizing polymer | 0.3-2% |

without enzymes, perfume, dyes; pH 7.5
Dosage 33 g/rinse cycle

When boric acid is used as the stabilizer compound, it is incorporated into the matrix at 1% and the pH is adjusted before the enzymes are incorporated.

When 4-FPBA is used as the stabilizer compound, it is added to the enzyme preparation at 2%.

If a peptide inhibitor is used as the stabilizer compound, it is added to the enzyme preparation at 0.5 mM. As an example of a peptide inhibitor, Cbz-Gly-Ala-Tyr-H was used here.

The enzymes, optionally mixed with an inhibitor, are incorporated into the dishwashing detergent matrix at an active enzyme content of 0.12%. The following proteases are used:
P1: Protease according to SEQ ID NO:5 from WO2017215925
P2: Coronase 48L (commercially available from Novozymes)
P3: Protease according to SEQ ID NO:2 from WO2011032988
P4: Protease according to SEQ ID NO:2 from WO2013060621

The dishwashing detergent matrix having the enzyme and inhibitor was stored at 40° C. for 1 week.

The activity of the protease is determined by the release of the chromophore para-nitroaniline (pNA) from the substrate succinyl-Ala-Ala-Pro-Phe-para-nitroanilide (AAPFpNA; Bachem L-1400). The release of the pNA causes an increase in absorbance at 410 nm, the temporal progression of which is a measure of the enzymatic activity. The measurement was carried out a temperature of 25° C., a pH of 8.6, and a wavelength of 410 nm. The measuring time was 5 minutes with a measuring interval of from 20 to 60 sec.

Measurement approach:
10 μl AAPF solution (70 mg/ml)
1,000 μl Tris/HCl (0.1 M, pH 8.6 with 0.1% Brij 35)
10 μl diluted protease solution
Kinetics over 5 min at 25° C. (410 nm)

Results for Protease Stabilizer Performance

The results after storage for 1 week at 40° C. are indicated in % stabilization, based on the residual activity of the composition without the addition of a stabilizer compound (also stored for 1 week at 40° C.):

| | | Peptide | Boric |
|---|---|---|---|
| Protease | 4-FPBA | inhibitor | acid |
| P1 | 312% | 186% | 223% |
| P2 | 173% | 111% | 160% |
| P3 | 170% | 107% | 169% |
| P4 | 125% | 89% | 136% |

It becomes clear that the protease P1 can be stabilized better by all three stabilizer compounds than the proteases P2, P3 or P4.

1. A washing or cleaning agent comprising:
(i) at least one protease having an amino acid sequence with at least 70% sequence identity with the amino acid sequence specified in SEQ ID NO:1 over the entire length thereof and has at least one amino acid substitution at at least one of the positions corresponding to the positions 12, 43, 122, 127, 154, 156, 160, 211, 212, or 222, in each case based on the numbering according to SEQ ID NO:1; and
(ii) at least one stabilizer compound selected from the group consisting of a phenylboronic acid derivative, boric acid, a peptide inhibitor, and combinations thereof.

2. The washing or cleaning agent according to claim 1, wherein the at least one amino acid substitution is selected from the group consisting of Q12L, I43V, M122L, D127P, N154S, T156A, G160S, M211N, M211L, P212D, P212H, or A222S, in each case based on the numbering according to SEQ ID NO:1.

3. The washing or cleaning agent according to claim 1, wherein the protease has one of the following amino acid substitution variants, in each case based on the numbering according to SEQ ID NO:1:
(i) I43V;
(ii) M122L, N154S, and T156A;
(iii) M211N and P212D;
(iv) M211L and P212D;
(v) G160S;
(vi) D127P, M211L, and P212D;
(vii) P212H; or
(viii) Q12L, M122L, and A222S.

4. The washing or cleaning agent according to claim 1, wherein the proportion by weight of the protease with respect to the total weight of the washing or cleaning agent, based on active protein, ranges from 0.005 to 5.0 wt. %.

5. The washing or cleaning agent according to claim 1,
wherein the peptide inhibitor is selected from the group consisting of a compound of formula (I), a compound of formula (II), and combinations thereof;
wherein the compound of formula (I) and/or the compound of formula (II) is optionally present together with a salt of formula (III);
wherein the compound of formula (I) has the following structural formula:
Z-A-NH—CH(R)—C(O)—X (I),
where A is an amino acid functional group; X is hydrogen; Z is an N-capping functional group selected from the group consisting of phosphoramidate [(R′O)₂(O)P—], sulfenamide [(SR′)₂—], sulfonamide [(R′(O)₂S—], sulfonic acid [SO₃H], phosphinamide [(R′)₂(O)P—], sulfamoyl derivatives [R′O(O)₂S—], thiourea [(R′)₂N(O)C—], thiocarbamate [R′O(S)C—], phosphonate [R′—P(O)OH], amidophosphate [R′O(OH)(O)P—], carbamate (R′O(O)C—), and urea (R′NH(O)C—);
where each R′ is independently selected from straight-chain or branched C₁-C₆ unsubstituted alkyl, phenyl, C₇-C₉ alkylaryl and cycloalkyl functional groups, where the cycloalkyl ring is optionally a C₄-C₈ cycloalkyl ring and optionally includes one or more heteroatoms selected from O, N, and S; and where R is selected from straight-chain or branched C₁-C₆ unsubstituted alkyl, phenyl and C₇-C₉ alkylaryl functional groups; and stereoisomers, tautomers and salts thereof;
wherein the compound of formula (II) has the following structural formula:
Y—B₁—B₀—X (II),
where X is hydrogen; B₁ is a single D or L amino acid functional group; B₀ is an amino acid functional group; and Y consists of one or more amino acid functional groups and optionally consists of an N-capping functional group, wherein the N-capping functional group is as defined under (I);
wherein the salt of formula (III) has the following structural formula:
(C^{E+})ₚ(D^{F-})_{q} (III),
where C is a cation selected from the group consisting of Al³⁺, Ca²⁺, Li⁺, Mg²⁺, Mn²⁺, Ni²⁺, K⁺, NR″₄⁺ and Na⁺, where each R″ represents, independently of one another, H or a linear or branched, substituted or unsubstituted alkyl group, aryl group or alkenyl group and optionally include one or more heteroatom(s); E is an integer from 1 to 3 and corresponds to the valency of the cation; p corresponds to the number of cations in the salt; D is an anion selected from the group consisting of CH₃COO⁻, Br⁻, CO₃²⁻, CI⁻, C₃H₅O(COO)₃³⁻, HCOO⁻, HCO₃⁻, HSO₄⁻, C₂O₄²⁻, SO₄²⁻, and SO₃²⁻; F is an integer from 1 to 3 and corresponds to the valency of the anion; q corresponds to the number of anions in the salt;
wherein the net charge on the salt is 0.

6. The washing or cleaning agent portion according to claim 5, wherein
A is selected from Ala, Gly, Val, Ile, Leu, Phe, and Lys;
R is selected from methyl, iso-propyl, sec-butyl, iso-butyl, —C₆H₅, —CH₂—C₆H₅, and —CH₂—CH₂—C₆H₅;
Z is selected from methyl, ethyl, or benzyl carbamate groups [CH₃O—(O)C—; CH₃CH₂O—(O)C—; or C₆H₅CH₂O—(O)C—], methyl, ethyl or benzylurea groups [CH₃NH—(O)C—; CH₃CH₂NH—(O)C—; or C₆H₅CH₂NH—(O)C—], methyl, ethyl or benzylsulfonamide groups [CH₃SO₂—; CH₃CH₂SO₂—; or C₆H₅CH₂SO₂—], and a methyl group, ethyl group or benzyl amidophosphate group [CH₃O(OH)(O)P—; CH₃CH₂O(OH)(O)P—; or C₆H₅CH₂O(OH)(O)P—];
B₀ is a D or L amino acid functional group selected from Tyr, m-tyrosine, 3,4-dihydroxyphenylalanine, Phe, Val, Met, Nva, Leu, Ile, and Nle;
B₁ is an amino acid functional group having an (optionally substituted) small aliphatic pendent group
Y is B₂, B₃-B₂, Z-B₂, Z-B₃-B₂, where B₂ and B₃ are, in each case independently of one another, an amino acid functional group and Z is an N-capping functional group, wherein the N-capping functional group is as defined in claim 1 under (I), where B₂ is selected from Val, Gly, Ala, Arg, Leu, Phe, and Thr, and/or B₃ is selected from Phe, Tyr, Trp, phenylglycine, Leu, Val, Nva, Nle and Iie; and
combinations thereof.

7. The washing or cleaning agent according to claim 1, wherein the phenylboronic acid derivative has the general structural formula (IV): 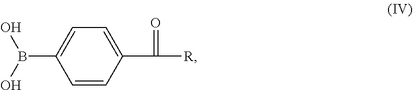
where R is hydrogen, a hydroxyl group, a C₁-C₆ alkyl group, a substituted C₁-C₆ alkyl group, a C₁-C₆ alkenyl group, or a substituted C₁-C₆ alkenyl group.

8. The washing or cleaning agent according to claim 1, wherein the phenylboronic acid derivative is 4-formylphenylboronic acid (4-FPBA).

9. The washing or cleaning agent according to claim 1,
wherein the stabilizer compound is selected from the group consisting of 4-FPBA, boric acid, peptide inhibitor, a combination of 4-FPBA and boric acid, a combination of 4-FPBA and peptide inhibitor, a combination of boric acid and peptide inhibitor, and a combination of 4-FPBA and boric acid and peptide inhibitor;
wherein the peptide inhibitor is selected from the group consisting of Cbz-Arg-Ala-Tyr-H, Ac-Gly-Ala-Tyr-H, Cbz-Gly-Ala-Tyr-H, Cbz-Gly-Ala-Tyr-H, Cbz-Val-Ala-Tyr-H, Cbz-Gly-Ala-Phe-H, Cbz-Gly-Ala-Val-H, Cbz-Gly-Gly-Tyr-H, Cbz-Gly-Gly-Phe-H, Cbz-Arg-Val-Tyr-H, Cbz-Leu-Val-Tyr-H, Ac-Leu-Gly-Ala-Tyr-H, Ac-Phe-Gly-Ala-Tyr-H, Ac-Tyr-Gly-Ala-Tyr-H, Ac-Phe-Gly-Ala-Leu-H, Ac-Phe-Gly-Ala-Phe-H, Ac-Phe-Gly-Val-Tyr-H, Ac-Phe-Gly-Ala-Met-H, Ac-Trp-Leu-Val-Tyr-H, MeO-CO-Val-Ala-Leu-H, MeNCO-Val-Ala-Leu-H, MeO-CO-Phe-Gly-Ala-Leu-H, MeO-CO-Phe-Gly-Ala-Phe-H, MeSO₂-Phe-Gly-Ala-Leu-H, MeSO₂-Val-Ala-Leu-H, PhCH₂O(OH)(O)P-Val-Ala-Leu-H, EtSO₂-Phe-Gly-Ala-Leu-H, PhCH₂SO₂-Val-Ala-Leu-H, PhCH₂O(OH)(O)P-Leu-Ala-Leu-H, PhCH₂O(OH)(O)P-Phe-Ala-Leu-H, MeO(OH)(O)P-Leu-Gly-Ala-Leu-H, α-MAPI, ß-MAPI, Phe-urea-Arg-Val-Tyr-H, Phe-urea-Gly-Gly-Tyr-H, Phe-urea-Gly-Ala-Phe-H, Phe-urea-Gly-Ala-Tyr-H, Phe-urea-Gly-Ala-Leu-H, Phe-urea-Gly-Ala-Nva-H, Phe-urea-Gly-Ala-Nle-H, Tyr-urea-Arg-Val-Tyr-H, Tyr-urea-Gly-Ala-Tyr-H, Phe-Cys-Ser-Arg-Val-Phe-H, Phe-Cys-Ser-Arg-Val-Tyr-H, Phe-Cys-Ser-Gly-Ala-Tyr-H, Antipain, GE20372A, GE20372B, chymostatin A, chymostatin B, and chymostatin C;
wherein the salt of formula (III) is Na₂SO₄; and
combinations thereof.

10. The washing or cleaning agent according to claim 1, wherein
when the at least one stabilizer compound is a peptide inhibitor, said peptide inhibitor is present in the washing or cleaning agent in an amount ranging from 0.01 to 15 wt. % based on the total weight of said washing or cleaning agent;
when the at least one stabilizer compound is a phenylboronic acid derivative, said phenylboronic acid derivative is present in the washing or cleaning agent in an amount ranging from 0.0005 to 2.0 wt. %, based on the total weight of said washing or cleaning agent;
when the at least one stabilizer compound is boric acid, said boric acid is present in the washing or cleaning agent in an amount ranging from 0.05 to 5.5 wt. %, based on the total weight of said washing or cleaning agent; and
combinations thereof.

11. The washing or cleaning agent according to claim 1, further comprising at least one further enzyme selected from the group consisting of amylases, cellulases, hemicellulases, mannanases, tannases, xylanases, xanthanases, xyloglucanases, β-glucosidases, pectinases, carrageenases, perhydrolases, oxidases, oxidoreductases, lipase, and combinations thereof.

12. The washing or cleaning agent according to claim 1, further comprising a dishwashing detergent.

13-15. (canceled)


SEQUENCE LISTING (20 sequences)

SEQ ID NO: 1 - protein, 269 aa, Bacillus gibsonii
QQTVPWGITRVQAPTVHNRGITGSGVKVAILDTGIAQHSDLTIRGGASFVPGESTTADLNGHGTHVAGTVAALNNSIGVIGVAPSADLYAVKVLGANGRGSVSGIAQGLEWAATNNMHIANMSLGSDAPSTTLERAVNYATSRGVLVIAATGNNGTGSIGYPARYANAMAVGATDQNNRRASFSQYGTGIDIVAPGVGIQSTYLNNSYASMPGTSMATPHVAGVAALVKQKNPSWNATQIRNHLKNTATNLGNSSQFGSGLVNADAATR

SEQ ID NO: 2 - protein, 4 aa, artificial sequence
  enzyme stabilizer
  MOD_RES (1)..(1): Ac-Leu, Ac-Phe, Ac-Tyr
XGAY

SEQ ID NO: 3 - protein, 4 aa, artificial sequence
  enzyme stabilizer
  MOD_RES (1)..(1): Ac-Phe
XGAL

SEQ ID NO: 4 - protein, 4 aa, artificial sequence
  enzyme stabilizer
  MOD_RES (1)..(1): Ac-Phe
XGAF

SEQ ID NO: 5 - protein, 4 aa, artificial sequence
  enzyme stabilizer
  MOD_RES (1)..(1): Ac-Phe
XGVY

SEQ ID NO: 6 - protein, 4 aa, artificial sequence
  enzyme stabilizer
  MOD_RES (1)..(1): Ac-Phe
XGAM

SEQ ID NO: 7 - protein, 4 aa, artificial sequence
  enzyme stabilizer
  MOD_RES (1)..(1): Ac-Trp
XLVY

SEQ ID NO: 8 - protein, 4 aa, artificial sequence
  enzyme stabilizer
  MOD_RES (1)..(1): MeO-CO-Phe, MeSO2-Phe, EtSO2-Phe, MeO(OH)(O)P-Leu
XGAL

SEQ ID NO: 9 - protein, 4 aa, artificial sequence
  enzyme stabilizer
  MOD_RES (1)..(1): MeO-CO-Phe
XGAF

SEQ ID NO: 10 - protein, 4 aa, artificial sequence
  enzyme stabilizer
  MISC_FEATURE (1)..(1): Phe-urea
XRVY

SEQ ID NO: 11 - protein, 4 aa, artificial sequence
  enzyme stabilizer
  MISC_FEATURE (1)..(1): Phe-urea
XGGY

SEQ ID NO: 12 - protein, 4 aa, artificial sequence
  enzyme stabilizer
  MISC_FEATURE (1)..(1): Phe-urea
XGAF

SEQ ID NO: 13 - protein, 4 aa, artificial sequence
  enzyme stabilizer
  MISC_FEATURE (1)..(1): Phe-urea
XGAY

SEQ ID NO: 14 - protein, 4 aa, artificial sequence
  enzyme stabilizer
  MISC_FEATURE (1)..(1): Phe-urea
XGAL

SEQ ID NO: 15 - protein, 4 aa, artificial sequence
  enzyme stabilizer
  MISC_FEATURE (1)..(1): Phe-urea
  MISC_FEATURE (4)..(4): Nva, Nle
XGAX

SEQ ID NO: 16 - protein, 4 aa, artificial sequence
  enzyme stabilizer
  MISC_FEATURE (1)..(1): Tyr-urea
XRVY

SEQ ID NO: 17 - protein, 4 aa, artificial sequence
  enzyme stabilizer
  MISC_FEATURE (1)..(1): Tyr-urea
XGAY

SEQ ID NO: 18 - protein, 6 aa, artificial sequence
  enzyme stabilizer
FCSRVF

SEQ ID NO: 19 - protein, 6 aa, artificial sequence
  enzyme stabilizer
FCSRVY

SEQ ID NO: 20 - protein, 6 aa, artificial sequence
  enzyme stabilizer
FCSGAY